(19) United States
(12) Patent Application Publication
(10) Pub. No.: US 2004/0242502 A1
(43) Pub. Date: Dec. 2, 2004

(54) Semi-synthetic saponin analogs with carrier and immune stimulatory activities for DNA and RNA vaccines

(75) Inventor: Dante J. Marciani, Birmingham, AL (US)

Correspondence Address: STERNE, KESSLER, GOLDSTEIN & FOX PLLC, 1100 NEW YORK AVENUE, N.W., WASHINGTON, DC, 20005, US

(73) Assignee: Galenica Pharmaceuticals, Inc., Birmingham, AL

(21) Appl. No.: 10/820,036

(22) Filed: Apr. 8, 2004

Related U.S. Application Data
(60) Provisional application No. 60/460,819, filed on Apr. 8, 2003.

(51) Int. Cl. A61K 31/704; C07H 15/24; C07J 17/00

(52) U.S. Cl. 514/26; 514/33; 536/6.1; 536/18.1

(57) ABSTRACT

The present invention discloses novel saponin derivatives for use with nucleic acids that induce an immune response when administered to animals and humans. The novel saponin derivatives disclosed comprise (a) a saponin aglycone core, wherein the aglycone core is covalently linked to one or more oligosaccharide chains; (b) a positively charged cationic chain, and optionally (c) a naturally occurring or synthetic lipophilic chain. Pharmaceutical and veterinary compositions comprising one or more of the novel saponin derivatives and saponin derivative/polynucleotide complexes are also disclosed. Disclosed as well are methods of using the novel saponin derivatives to deliver a polynucleotide molecule to cells of an animal, to stimulate or generate an immune response in an animal, and to generate a detectable immune response in an animal.

### CROSS-REFERENCE TO RELATED APPLICATIONS

This application claims the benefit, under 35 U.S.C. § 119(e), of the earlier filing date of U.S. Provisional Application No. 60/460,819, filed on Apr. 8, 2003, the contents of which is fully incorporated by reference herein in its entirety.

### BACKGROUND OF THE INVENTION

1. Field of the Invention

The present invention is in the field of nucleic acid and antisense nucleic acid delivery into cells. More particularly, the invention pertains to novel saponin derivatives for use with nucleic acids that induce an immune response when administered to animals and humans.

2. Background Art

DNA and RNA vaccines are the terms broadly used to describe methods of transiently transfecting cells with DNA plasmids or mRNA encoding for protein antigens whose expression stimulates an immune response. Because of the intracellular production of these antigens and their processing by the endogenous pathway, nucleic acid vaccines elicit humoral as well as T-cell immunity with cytotoxic T lymphocytes (CTL) production.

The immune system may exhibit both specific and nonspecific immunity (Klein, J., et al., Immunology (2nd), Blackwell Science Inc., Boston (1997)). Generally, specific immunity is produced by B and T lymphocytes, which display specific receptors on their cell surface for a given antigen. The immune system may respond to different antigens in two ways: 1) humoral-mediated immunity, which includes B cell stimulation and production of antibodies or immunoglobulins (other cells, however, are also involved in the generation of an antibody response, e.g. antigen-presenting cells (APCs, including macrophages) and helper T cells (Th1 and Th2)), and 2) cell-mediated immunity (CMI), which generally involves T cells, including cytotoxic T lymphocytes (CTLs), although other cells are also involved in the generation of a CTL response (e.g., Th1 and/or Th2 cells and APCs).

Nonspecific immunity encompasses various cells and mechanisms such as phagocytosis (the engulfing of foreign particles or antigens) by macrophages or granulocytes, and natural killer (NK) cell activity, among others. Nonspecific immunity relies on mechanisms less evolutionarily advanced (e.g., phagocytosis, which is an important host defense mechanism) and does not display the acquired nature of specificity and memory, hallmarks of a specific immune response.

Stimulation of an immune response is not limited to DNA plasmids or mRNA encoding for protein antigens. Non-coding bacterial DNA and oligonucleotides containing CpG motifs have also been shown to stimulate immunity (Yamamoto, S., et al., Microbiol. Immunol. 36:983-997 (1992); Hacker, G., et al., Immunology 105:245-251 (2002)).

DNA and RNA vaccines should elicit strong humoral and T-cell immune responses. However, in many cases the responses are not as strong as desired. This may be due to the ineffective targeting of antigen presenting cells (APC), such as macrophages and dendritic cells, by the DNA plasmids or RNA. A lack of targeting results in a significant transfection of other cells, such as myocytes, whose low class I major histocompatibility complex (MHC-1) levels and lack of costimulatory molecules such as B7 make them poor candidates for stimulation of antibodies or CTL. In effect, it has been shown that delivery of DNA to APC results in a rapid CTL induction and the production of higher avidity antibodies (Boyle, J. S., et al., Proc. Nat. Acad. Sci. USA 94:14626-14631(1997)). However, the quality of the immune response stimulated by these vaccines also depends on the recipient immune system's competence. Thus, compromised or weakened immune systems, such as those found in cancer patients and the elderly, might fail to mount an effective protective immune response without the help of one or more immune stimulants. In general, experimental DNA viral vaccines confer immunity on roughly half of the animals immunized, indicating the need for both APC targeting and immune stimulation.

Different procedures have been devised to avoid the limitations caused by the lack of targeting by DNA sequences (Lasic, D. D., Liposomes in Gene Delivery, CRC Boca Raton, 1997). In some cases, DNA plasmids have been enclosed in conventional liposomes to target macrophages. In other cases, the DNA or RNA has been mixed with positively charged polymers to form complexes that are supposed to be taken up by APCs. In still others, the positively charged polymers have been conjugated to lipid chains, cholesterol or steroids, to facilitate the uptake of these nucleic acid complexes by cells via endocytosis, to avoid the lysosomal compartment and the concomitant nucleic acid degradation. Because classic liposomes do not significantly increase the intracellular delivery of nucleic acids, liposomes containing cationic lipids have been used instead. For example, enclosure of bacterial DNA or CpG oligonucleotides in liposomes containing cationic lipids has been shown to enhance their immunostimulatory properties (Yamamoto, T., Microbiol. Immunol. 38:831-836 (1994); Dow, S. W., et al., J. Immunol. 163:1552-1561 (1999; and Siders, W. F., Mol. Ther. 6:519-527 (2002)). Cationic lipids can form complexes with DNA that are able to transfect cells. However, cationic lipids have damaging effects on biological systems. For instance, they can induce platelet aggregation, hemolysis, cytotoxicity, and other damaging effects. This may limit their use to research only.

Therefore, there is a need for additional agents that either increase the amount of transfection or the degree of immune stimulation that occurs upon administration of a DNA or RNA vaccine.

The inventions described herein address these needs by providing novel, effective compounds that i) facilitate the targeting and delivery of DNA or RNA to the APCs' cytosol, i.e. act as carriers, and/or ii) co-stimulate the immune system to produce an effective response, preferentially that of a Th1 type, i.e. to act as immune stimulants.

Saponins are glycosidic compounds that are produced as secondary metabolites. They are widely distributed among higher plants and in some marine invertebrates of the phylum Echinodermata (ApSimon et al., Stud. Org. Chem. 17:273-286 (1984)). Because of their antimicrobial activity, plant saponins are effective chemical defenses against microorganisms, particularly fungi (Price et al., CRC Crit. Rev. Food Sci. Nutr. 26:27-135 (1987)). Saponins are responsible for the toxic properties of many marine invertebrates (ApSimon et al., Stud. Org. Chem. 17:273-286 (1984)). The chemical structure of saponins imparts a wide range of pharmacological and biological activities, including some potent and efficacious immunological activity. In addition, members of this family of compounds have foaming properties (an identifying characteristic), surfactant properties (which are responsible for their hemolytic activity), cholesterol-binding, fungitoxic, molluscicidal, contraceptive, growth-retarding, expectorant, antiinflammatory, analgesic, antiviral, cardiovascular, enzyme-inhibitory, and antitumor activities (Hostettmann, K., et al., Methods Plant Biochem. 7:435-471(1991); Lacaille-Dubois, M. A. & Wagner, H., Phytomedicine 2:363-386 (1996); Price, K. R., et al., CRC Crit. Rev. Food Sci. Nutr. 26:27-135 (1987)).

Structurally, saponins consist of any aglycone (sapogenin) attached to one or more sugar chains. In some cases saponins may be acylated with organic acids such as acetic, malonic, angelic and others (Massiot, G. & Lavaud, C., Stud. Nat. Prod. Chem. 15:187-224(1995)) as part of their structure. These complex structures have molecular weights ranging from 600 to more than 2,000 daltons. The asymmetric distribution of their hydrophobic (aglycone) and hydrophilic (sugar) moieties confers an amphipathic character to these compounds which is largely responsible for their detergent-like properties. Consequently, saponins can interact with the cholesterol component of animal cell membranes to form pores that may lead to membrane destruction and cell death, such as the hemolysis of blood cells.

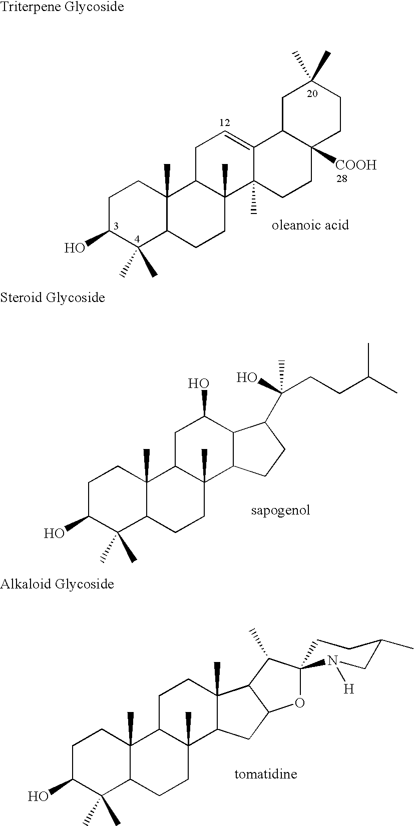

Saponins can be classified according to their aglycone composition as shown above:

Triterpene glycosides

Steroid glycosides

Steroid alkaloid glycosides

The steroid alkaloid glycosides, or glycoalkaloids, share many physical and biological properties with steroid glycosides, but alkaloid glycosides are usually considered separately because their steroidal structure contains nitrogen. Frequently, the aglycones have methyl substituents that may be oxidized to hydroxymethyl, aldehyde or carboxyl groups; these moieties may play a role in some of the saponins' biological activities. From extensive studies of saponins, it is apparent that the triterpene saponins are not only the most predominant in nature, but also those with the most interesting biological and pharmacological properties.

Saponins have one or more linear or branched sugar chains attached to the aglycone via a glycosidic ether or ester link. In some saponins, the presence of acylated sugars has also been detected. According to the number of sugar chains attached to the aglycone, the saponins can be monodesmosidic saponins (with a single sugar chain), or bidesmosidic saponins (with two sugar chains). In the monodesmosidic saponins, the sugar chain is typically attached by a glycosidic ether linkage at the C-3 of the aglycone. In addition to the C-3 linked sugar chain, bidesmosidic saponins have a second sugar chain bound at C-28 (triterpene saponins) or at C-26 (steroid saponins) by an ester linkage. Because of the typical lability of esters, bidesmosidic saponins are readily converted into their monodesmosidic forms by mild hydrolysis (Hostettmann, K., et al., Methods Plant Biochem. 7:435-471 (1991)).

Saponins from the bark of the Quillaja saponaria Molina tree (quillaja saponins) are chemically and immunologically well-characterized products (Dalsgaard, K. Arch. Gesamte Virusforsch. 44:243 (1974); Dalsgaard, K., Acta Vet. Scand. 19 (Suppl. 69):1 (1978); Higuchi, R. et al., Phytochemistry 26:229 (1987); ibid. 26:2357 (1987); ibid. 27:1168 (1988); Kensil, C. et al., J. Immunol. 146:431 (1991); Kensil et al., U.S. Pat. No. 5,057,540 (1991); Kensil et al., Vaccines 92:35 (1992); Bomford, R. et al., Vaccine 10:572 (1992); and Kensil, C. et al., U.S. Pat. No. 5,273,965 (1993)). From an aqueous extract of the bark of the South American tree, with Quillaja saponaria Molina, twenty-two peaks having saponin activity were separated by chromatographic techniques. The predominant purified saponins were identified as QS-7, QS-17, QS-18 and QS-21. QS-21 was later resolved into two additional peaks, each comprising a discrete compound, QA-21-V1 and QA-21-V2. See Kensil et al., U.S. Pat. No. 5,583,112 (1996).

These saponins are a family of closely related O-acylated triterpene glycoside structures. They have an aglycone triterpene (quillaic acid), with branched sugar chains attached to positions 3 and 28, and an aldehyde group in position 4. Quillaja saponins have an unusual fatty acid substituent (3,5-dihydroxy-6-methyloctanoic acid) as a diester on the fucose residue of the C-28 carbohydrate chain. This ester is hydrolyzed under mildly alkaline conditions or even at physiological pH over short periods of time to produce deacylated saponins, including DS-1 and DS-2 (Higuchi et al., Phytochemistry 26:229 (1987)); (Kensil et al., Vaccines 92:35-40 (1992)). More severe hydrolysis of these saponins using strong alkalinity (Higuchi et al., Phytochemistry 26:229 (1987)) or prolonged hydrolysis (Pillion, D. J., et al., J. Pharm. Sci., 85:518-524 (1996)) produces QH-957, the result of hydrolysis of the C-28 ester. The triterpenoid hydrolysis by-products have hydrophobic/hydrophilic properties differing from those of QS-21; these differences result in altered micellar and surfactant properties.

Some saponins have been shown to have different types of immune stimulating activities, including adjuvant activity. These activities have been reviewed previously (Shibata, S., New Nat. Prod. Plant Pharmacol. Biol. Ther. Act., Proc. Int. Congr. 1st, 177-198 (1977); Price, K. R., et al., CRC Crit. Rev. Food Sci. Nutr. 26:27-135 (1987); Schopke, Th. and Hiller, K., Pharmazie 45:313-342 (1990); Lacaille-Dubois, M. A., et al., Phytomedicine 2:363-386 (1996); Press, J. B. et al., Stud. Nat. Prod. Chem. 24:131-174 (2000)). Immune adjuvants are compounds that, when administered to an individual, increase the immune response to an antigen in a test subject to which the antigen is administered, or enhance certain activities of cells from the immune system. Immune adjuvants modify or immunomodulate the cytokine network, up-regulating the humoral and cellular immune response. Humoral response elicits antibody formation. Cellular immune response involves the activation of T cell response, Th1 or Th2, to mount this immune response. Th1 responses will elicit complement fixing antibodies and strong delayed-type hypersensitivity reactions associated with IL-2, IL-12, and γ-interferon. Induction of cytotoxic T lymphocytes (CTLs) response also appears to be associated with a Th1 response. Th2 responses are associated with high levels of IgE, and the cytokines IL-4, IL-5, IL-6, and IL-10. The aldehyde-containing saponins such as those from quillaja induce a strong Th1 response. However, some of their analogs may induce a Th2 response.

Saponin adjuvants can target different cells, i.e., macrophages, dendritic cells, hepatocytes, and others, by binding via their glycosyl residues to specific cell surface receptors. The saponins' triterpene or steroid moieties, by interacting with the cholesterol containing cell membrane lipid bilayer, allow the delivery of compounds complexed with the saponins directly to the cells' cytosol. Addition of a lipid side-chain to saponins results in a significant enhancement of this capacity. See Marciani, D. J., U.S. Pat. No. 5,977,081 (1999). Saponins containing an aldehyde, by reacting with amino groups of receptor protein(s) present on certain T-cells and forming Schiff bases, stimulate Th1 immunity. Although saponins are effective adjuvants for proteins and carbohydrate antigens, they are not good carriers and/or stimulants of immunity when used in conjunction with DNA or RNA vaccines.

Novel cationic compounds have been synthesized by Ren et al. (Tetrahedron Letts. 42:1007-1010 (2001)) which contain trivalent galactosides that act to target specific cells for more effective transfection of DNA.

### BRIEF SUMMARY OF THE INVENTION

The present invention is directed to novel saponin derivatives comprising:

(a) a saponin aglycone core, wherein the aglycone core is covalently linked to one or more oligosaccharide chains; and

(b) a positively charged cationic chain, wherein the cationic chain comprises (i) three or more carbon atoms; and (ii) one or more primary, secondary, or tertiary amine groups, or one or more guanidine groups, or any combination thereof; and wherein the cationic chain is covalently bound either to the aglycone core or to one or more oligosaccharide chains of the derivative. The saponin derivative may further comprise (c) a naturally occurring or synthetic lipophilic chain, wherein the lipophilic chain comprises from 4 to 36 carbon atoms and optionally contains one or more oxyethylene groups.

The present invention is also directed to pharmaceutical and veterinary compositions comprising one or more of the saponin derivatives and one or more pharmaceutically acceptable diluents, carriers or excipients.

The present invention is further directed to a saponin derivative/polynucleotide complex comprising one or more of the saponin derivatives associated with a polynucleotide molecule. In this embodiment of the invention, the polynucleotide molecule is a non-coding bacterial DNA, or either DNA or RNA that at least partially encodes a peptide or polypeptide antigen. Useful antigens are peptide or polypeptide antigens associated with a pathogen such as a bacterium or virus that causes illness in a human or animal; or antigens associated with the presence of cancer in a human or animal.

The present invention is also directed to a saponin derivative/polynucleotide secondary complex comprising one or more saponin derivative/polynucleotide complexes described above in admixture or associated with one or more saponins selected from the group consisting of a native saponin, a semi-synthetic saponin derivative, and a synthetic saponin containing a triterpenoid aglycone core covalently linked to one or more oligosaccharide chains.

The present invention is further directed to pharmaceutical compositions comprising one or more saponin derivatives, a polynucleotide, and a pharmaceutically acceptable carrier or diluent; to a method of making the primary and secondary complexes described above; and to a method of making products produced by such methods.

The present invention is still further directed to a method of delivering a polynucleotide to cells of an animal in need thereof, comprising administration in vivo to an animal of a polynucleotide construct comprising a polynucleotide sequence encoding an immunogen, and one or more of the saponin derivatives of the invention. In this embodiment of the invention, the polynucleotide sequence can be either DNA or RNA. If the polynucleotide sequence is DNA, the sequence may be operably linked to a promoter.

The present invention is also directed to a method of delivering a polynucleotide to cells of an animal in need thereof, comprising the steps of (a) forming a saponin derivative/polynucleotide complex, wherein the complex is comprised of one or more of the saponin derivatives of the invention associated with a polynucleotide sequence encoding an immunogen;

and (b) administering the complex in vitro to the cells of the animal in an amount sufficient that uptake of said polynucleotide sequence into the cells of the animal occurs. In this embodiment of the invention, the polynucleotide sequence can be either DNA or RNA. If the polynucleotide sequence is DNA, the sequence may be operably linked to a promoter.

The present invention is further directed to a method of stimulating or generating an immune response in an animal in need thereof, comprising administering in vivo to the animal a polynucleotide sequence encoding an immunogen, and one or more of the saponin derivatives of the invention, in an amount sufficient that uptake of the polynucleotide sequence into cells of the animal occurs, and sufficient expression results, to stimulate or generate the immune response in the animal. In this embodiment of the invention, the polynucleotide sequence can be a DNA sequence linked to a promoter.

The present invention is also directed to a method of stimulating or generating an immune response in an animal in need thereof, comprising administering in vivo to the animal a noncoding bacterial DNA polynucleotide and one or more of the saponin derivatives of the invention, to stimulate or generate the immune response in the animal. The method can further comprise administering in vivo to the animal a polypeptide antigen, or a polynucleotide sequence encoding an immunogen.

The present invention is also directed to a method of stimulating or generating an immune response in an animal in need thereof, comprising the steps of (a) introducing into the cells of the animal a polynucleotide sequence encoding an immunogen, and one or more of the saponin derivatives of the invention; and (b) introducing the cells into the animal, wherein sufficient expression of the immunogen occurs in the cells and an immune response is stimulated or generated in the animal. In this embodiment of the invention, the polynucleotide sequence can be a DNA sequence that is operably linked to a promoter.

The present invention is also directed to a method of generating a detectable immune response in an animal in need thereof, comprising administering in vivo to the cells of an animal a polynucleotide sequence encoding an immunogen, and one or more of the saponin derivatives of the invention, in an amount sufficient that uptake of the polynucleotide sequence into the cells of the animal occurs, and sufficient expression results, to generate the detectable immune response. In this embodiment of the invention, the polynucleotide sequence can be a DNA sequence that is operably linked to a promoter.

The present invention is further directed to a method of generating a detectable immune response in an animal in need thereof, comprising the steps of (a) introducing into the cells of the animal a polynucleotide sequence encoding an immunogen, and one or more of the saponin derivatives of the invention; and (b) introducing the polynucleotide sequence into the cells into the animal, wherein sufficient expression of the immunogen occurs in the cells and a detectable immune response is generated. In this embodiment of the invention, the polynucleotide sequence can be a DNA sequence that is operably linked to a promoter.

### BRIEF DESCRIPTION OF THE FIGURES

FIG. 1 illustrates the effects of DMPS (3-dimethylamino-1-propylamino-DS-saponin derivative (compound III in Scheme 1a)) on the immune response of individual female Balb/c mice to OVA DNA.

FIG. 2 illustrates the effects of DMPS on the immune response of female Balb/c mice to OVA DNA, expressed as average values of absorbance at 450 nm.

FIG. 3 illustrates the effects of DMPS (GPI-0330) on the IgG1 and IgG2a response to OVA DNA vaccination.

FIG. 4. illustrates the effects of the polyethylenimine quillaja saponin derivative of Example 5d (GPI-0332) on the IgG1 and IgG2a response to OVA DNA vaccination.

### DETAILED DESCRIPTION OF THE INVENTION

The present invention is directed to novel saponin derivatives comprising:

(a) an aglycone core substituted with one or more oligosaccharide chains; and

(b) a positively charged cationic chain comprising (i) three or more carbon atoms, and (ii) one or more primary, secondary, or tertiary amine groups, or one or more guanidine groups, or any combination thereof. The novel saponin derivatives may optionally include a naturally occurring or synthetic lipophilic chain covalently attached to either the aglycone core or to one or more of the oligosaccharide chains.

Appropriate saponins include triterpene glycosides, steroid glycosides, and steroid alkaloid glycosides, with triterpene glycosides the most preferred saponins. Thus a preferred aglycone core is a triterpenoid aglycone core.

One or more oligosaccharide chains may be covalently liked to the aglycone core. If the aglycone core is a triterpene nucleus, there are preferably one or two oligosaccharide chains linked at positions 3 and/or 28 of the triterpene nucleus. The attached oligosaccharide chains are capable of binding to carbohydrate receptors on the cells' surface, preferentially of APCs, such as macrophages and dendritic cells.

The saponin derivative may have an aldehyde or ketone group, preferably an aldehyde group, in its aglycone or its oligosaccharide chains that is capable of forming an imine or Schiff base with an amino group. The formation of an imine or Schiff base with certain cell surface receptors, preferentially on an APC, provides a co-stimulatory signal needed for stimulation of an immune response, preferentially of type Th1. If the aldehyde or ketone group is attached to the aglycone core, the aldehyde or ketone group will be attached preferably at position 4 of the core.

Quillaja, Gypsophila and Saponaria are saponins useful in the present invention, all having triterpene aglycones with an aldehyde group linked or attached to position 4, branched oligosaccharides linked by an ester bond in position 28, and a 3-O-glucuronic acid (3-O-glcA) that in Quillaja and Gypsophila is linked to branched oligosaccharides. Saponins from Q. saponaria and S. jenisseenis include acyl moieties, whereas saponin from Gypsophila, Saponaria, and Acanthophyllum do not include acyl moieties. Each of these non-acylated or de-acylated saponins is useful in the present invention. Saponins without aldehyde groups, such as soyasaponins, camellidin, and dubioside F, are also useful in the present invention.

Other triterpene saponins are suitable for use in the present invention and include, for example, the bidesmosidic saponin, squarroside A, isolated from Acanthophyllum squarrosum; the saponin lucyoside P; and two acylated saponins isolated from Silene jenisseensis Willd. See, for example, U.S. Pat. No. 6,080,725.

Attached to the saponin derivative is a positively charged cationic chain, which is covalently bound to either the aglycone core or to a sugar residue of an oligosaccharide chain of the saponin derivative. The cationic chain can have a molecular weight of 100 to 100,000 daltons and may have one or more positively charged cationic groups. For purposes of the present invention, the cationic group must possess a positive charge under particular environmental or physiological conditions. Thus, amine groups are considered to be cationic since amine groups are protonated under a variety of environmental and physiological conditions.

In the present invention, the cationic chain can be any cationic amine capable of being linked to the aglycone core or to a sugar residue. Thus the cationic chain must contain at least one of the following chemical groups: a carboxyl group, a primary or secondary amine group, a thiol group, a hydroxyl group, or a chemical group capable of being activated to form a covalent bond to the aglycone and/or sugar moieties of a saponin. See Behr et al. (Proc. Natl. Acad. Sci. 86:6982-6986 (1989)) and Wheeler (U.S. Pat. No. 5,861,397) for examples of cationic chains that are suitable for use in the present invention.

Preferably, the cationic chain comprises (i) a minimum of three (3) carbon atoms and (ii) contains one or more primary, secondary, or tertiary amine groups, or one or more guanidine groups, or any combination thereof.

The cationic chain can be a linear or branched aliphatic chain. Examples of cationic chain aliphatic groups include straight-chained or branched, saturated or unsaturated aliphatic groups having about 3 to about 24 carbon atoms, preferably 3 to 20 carbon atoms, more preferably 3 to 16 carbon atoms, and most preferably 6 to 12 carbon atoms. Examples of useful aliphatic groups include pentyl, hexyl, heptyl, octyl, nonyl, decyl, dodecyl, and hexadecyl. Examples of preferred aliphatic amine cationic chains include 3-dimethylamino-1-propylamino, 3-trimethylamino-1-propylamino, 5-dimethyl-1-pentylamino; polyamine chains having 10-16 atoms, such as spermine and spermidine; aliphatic chains containing one or more pyridinium, pyrimidinium, or imidazolinium groups; and choline.

Additional examples of preferred cationinc chains include linear and branched polyethylenimines, glucosamine, and mannosamine.

The positively charged cationic chain can also be an oligosaccharide or a polysaccharide; a protein, such as a histone or protamine; or a synthetic or natural oligopeptide or polypeptide with a series of basic amino acids, i.e. lysine and arginine, such as a polylysine chain. The positively charged cationic chain can also be a polypeptide that is cationic or has been subsequently modified by the introduction of amino groups or similar cationic basic groups that are capable of forming a complex with DNA or RNA.

Proteins and polypeptides can be modified by introducing cationic groups using one of the following methods: i) introducing an amine group at the carboxyl group of a protein or polypeptide by reaction with a diamine (e.g., ethylenediamine, Jeffamine EDR-148) using carbodiimide mediated coupling, with active ester intermediates such as NHS esters, or with agents such as N,N′-carbodiimidazole; ii) creating a carboxylate group from a hydroxyl group by reaction with choloroacetic acid. The new carboxyl group can be modified by reaction with a diamine as previously described; iii) modifying sulfhydryl groups with N-(β-iodoethyl)trifluoroacetamide to yield an intermediate that undergoes spontaneous deblocking, yielding an aminoethyl derivative linked via a thioether; iv)modifying sulfhydryls with ethylenimine or with 2-bromoethylamine to yield an aminoethyl derivative (see Hermanson, G. T., Bioconjugate Techniques, Academic Press, New York, 1996); v) converting a sulfhydryl group to a basic derivative, 4-thialaminine, by alkylation with (2-bromoethyl)trimethylammonium; vi) treating a protein with O-methylisourea at alkaline pH to convert primary amino groups to the more basic guanidinium groups, i.e., changing the lysyl residues to homoarginine derivatives.

The saponin derivative, in addition to the positively cationic charged chain, may optionally have a lipophilic chain. This lipophilic chain may be linked to the aglycone core or to a sugar residue of an oligosaccharide chain of the saponin derivative. The lipophilic chain comprises 4 to 36 carbon atoms, preferably 10 to 14 carbon atoms, and most preferably 12 carbon atoms, and may be linear or branched, and saturated or unsaturated and may optionally contain one or more oxyethylene groups. By way of example, useful lipophilic chains include fatty acids, terpenes, polyethylene glycols, and linear or branched lipid chains. Additional useful lipophilic chains include those described in U.S. Pat. No. 6,262,029 (“Chemically Modified Saponins and the Use Thereof as Adjuvants”), at columns 7 to 11. Lipophilic chains suitable for use in the present invention do not contain cationic groups such as primary, secondary or tertiary amine groups.

Useful fatty acid lipophilic chains include C₆-C₂₄ fatty acids, preferably C₇-C₁₈ fatty acids. Examples of preferred useful fatty acids include saturated fatty acids such as lauric, myristic, palmitic, stearic, arachidic, behenic, and lignoceric acids; and unsaturated fatty acids, such as palmitoleic, oleic, linoleic, linolenic and arachidonic acids.

Useful terpenoids include retinol, retinal, bisabolol, citral, citronellal, citronellol and linalool.

Useful polyethylene glycols have the formula H—(O—CH₂—CH₂)ₙOH, where n, the number of ethylene oxide units, is from 4 to 14. Examples of useful polyethylene glycols include PEG 200 (n=4), PEG 400 (n=8-9), and PEG 600 (n=12-14). Useful polyethylene glycol fatty alcohol ethers include those wherein the ethylene oxide units (n) are between 1 to 8, and the alkyl group is from C₆ to C₁₈.

In a first preferred embodiment of this invention, the positively charged cationic chain of the saponin derivative is covalently linked to one of the glycosyl residues of the saponin, preferentially to a carboxylic group, such as that present on glucuronic and galacturonic acid residues.

For compounds of this embodiment, the cationic chain can be linked to a carboxyl group via one of their primary amino groups using the carbodiimide reaction in the presence of N-hydroxysuccinimide (NHS) or their water-soluble analogs. See Schemes 1a-1c and the syntheses described in Example 1 below. For small cationic chains (C3 to C18) carrying 2 to or more amino groups, the reaction is carried out in the presence of an excess of the cationic chain, to avoid the incorporation of multiple saponin groups to the chain. For large cationic chains, (polylysine, protamines and others), the number of saponin residues per chain can be adjusted by increasing or decreasing the relative proportions of saponin and cationic chain. In both cases, the resulting compounds would have saponin residues that might or might not carry aldehyde groups. These derivatives would not carry a lipophilic side-chain.

Thus, saponin derivatives of this embodiment include a compound of Formula I:

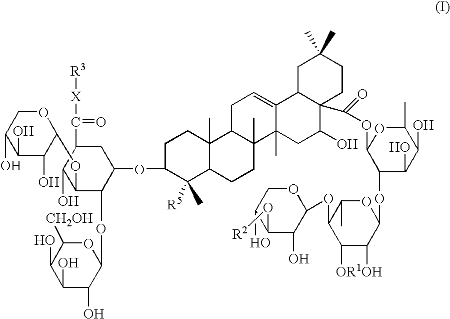

or a pharmaceutically acceptable salt thereof, wherein

R¹ is glucose or hydrogen;

R² is apiose or xylose, preferably apiose;

X is —NH;

R³ is C₄-C₃₀ alkyl or C₄-C₃₀ alkenyl, either of which is optionally substituted by one or more —NH₂, —NHR⁴, —N(R⁴)₂, —NH₃⁺, —(NH₂R⁴)⁺, —N(R⁴)₃⁺, or —NH((R⁴)₂N)C(═NR⁴), where R⁴ is hydrogen or lower alkyl; and either of which is optionally interrupted by one or more NH, NH₂⁺, S, O, C═O, or NR⁴ groups; or

R³ is an oligosaccharide, polysaccharide, or protein; and

R⁵ is CH═O, CH₃, or CH₂OH.

In a preferred aspect, R³ is selected from the group consisting of a C₄-C₃₀ straight or branched chain alkyl group, and a C₄-C₃₀ straight or branched chain alkenyl group; either of which is optionally substituted by one or more —NH₂, —NHR₄, —N(R⁴)₂, or —NH(H₂N)C(═NH)—, where R⁴ is hydrogen or a lower alkyl, preferably methyl, and either of which is optionally interrupted by one or more NH groups. Preferred R³ groups in this aspect include aliphatic amines and polyamines.

In a second preferred aspect, R³ is an oligosaccharide, polysaccharide, or protein. Preferred oligosaccharides and polysaccharides include those composed mostly of amino sugars such as glucosamine and mannosamine, or chemically aminated sugars.

Non-limiting examples of saponin derivatives of this first embodiment include compound III of Scheme 1a and compound VIII of Scheme 1c.

In a second preferred embodiment of the invention, the saponin derivatives have a positively charged cationic chain attached to the aldehyde group on the aglycone nucleus of the saponin.

Thus, compounds of this second embodiment include compounds of Formula I wherein:

X is —O—; R³ is H;

R⁵ is —CHNHR⁶, wherein R⁶ is C₄-C₃₀ alkyl or C₄-C₃₀ alkenyl, either of which is optionally substituted by one or more —NH₂, —NHR₄, —N(R⁴)₂, —NH₃⁺, —(NH₂R⁴)⁺, —N(R⁴)₃⁺, or —NH((R⁴)₂N)C(═NR⁴), where R⁴ is hydrogen or lower alkyl; and either of which is optionally interrupted by one or more NH or NH₂⁺ groups; or R⁶ is an oligosaccharide or polysaccharide, preferably an oligosaccharide or polysaccharide composed of aminated sugars, or R⁶ is a protein; and

R¹ and R² have the same definitions are indicated above for compounds of the first embodiment. Examples of compounds in this embodiment are presented in Scheme 2.

For compounds in this second embodiment, the cationic chain can be linked to the aldehyde using reductive amination in the presence of Na cyanoborohydride or Na borohydride. See, for example, the synthesis outlined in Scheme 2a and in Example 2 below. As for compounds of the first embodiment (Formula I), the number of saponin residues per cationic chain can be selected by adjusting the relative proportions of the glycoside or saponin and the cationic polymer. Because the aldehyde group will be used during the reaction with the primary amine, the resultant saponin derivative does not have the capacity to co-stimulate T-cells. Thus, compounds of this second embodiment of the invention can be used in combination with natural saponins, semi-synthetic or synthetic saponin derivatives carrying aldehyde groups. Formation of micelles between the cationic chain-saponin derivative/polynucleotide complex and the aldehyde-carrying saponin will deliver co-stimulatory signals to the T-cells.

Non-limiting examples of compounds of this second embodiment include compound X of Scheme 2a.

In a third embodiment of the invention, the saponin derivatives have structures similar to that described for compounds of the first two embodiments, but a lipophilic chain is attached to the saponin residues of these derivatives. For those compounds of this embodiment in which the cationic chain is linked to a sugar residue, the lipophilic chain can be added at the aldehyde group of the aglycone nucleus by reductive amination with an alkyl monoamine, such as dodecylamine. See, for example, Scheme 3a and Example 3 below. For those compounds in which the positively charged cationic chain attached to the aldehyde group of the aglycone nucleus, the lipophilic chain can be added to a sugar residue, such as glucuronic acid, by reacting with an alkyl monoamine in the presence of carbodiimide and NHS. In both cases, the corresponding derivatives lack or have a limited number of aldehyde residues. If co-stimulation is required, then the nucleic acid complexes formed with these derivatives must be used in combination with aldehyde-carrying native saponins or their semi-synthetic derivatives, such as GPI-0 100.

Non-limiting examples of compounds of this embodiment include compound XIII of Scheme 3a below.

In a fourth embodiment of the invention, the saponin derivatives have a lipophilic chain which is attached to a sugar residue of the oligosaccharide chain of the saponin, preferentially to the carboxyl group of a glucuronic or galacturonic acid. Scheme 4a outlines the synthetic steps for preparing a compound of this embodiment.

Non-limiting examples of compounds of this embodiment include compound XIV in Scheme 4a below.

In a fifth embodiment of the invention, the cationic chain of the saponin derivatives is a protein or a polymer. Compounds of this embodiment include conjugates between a saponin (such as desacylated quillaja saponin, gypsophylla saponin, and other similar glycosides or saponins) and i) a protein such as protamine or histone, or ii) a polymer such as polylysine, polyethylenimine, polyglucosamine or chitosan. The polymers are linked to the glycoside or saponin moiety by either the carboxyl or aldehyde groups. Schemes 5a-5c outline the synthetic steps required for preparation of three compounds of this fifth embodiment.

Non-limiting examples of compounds of this fifth embodiment include compound XV in Scheme 5a and compound XVIII in Scheme 5b below. Preparation of the Saponin Derivatives

Cationic saponin derivatives of the present invention can be synthesized from saponin starting materials using conventional synthetic protocols known to those of ordinary skill in the art. See, for example, U.S. Pat. No. 6,080,725, for a description of synthetic protocols used in the preparation of desacylsaponin starting materials.

Schemes 1a-5c and Examples 1-5 herein provide synthetic protocols for the preparation of specific cationic saponin derivatives of the invention. One of ordinary skill in the art will know how to use these synthetic protocols, and adapt them when necessary, to prepare additional saponin derivatives falling within the scope of the invention.

Use of the Saponin Derivatives with DNA and RNA Polynucleotides

The saponin derivatives of the present invention can be combined with DNA or RNA polynucleotides and used to enhance the immune response of an animal or to stimulate or generate an immune response in an animal. For example, the saponin derivatives can be used with coding or noncoding bacterial DNA, plasmid DNA, polynucleotides or CpG oligonucleotides to stimulate a non-specific innate immune response in an animal. The term “noncoding bacterial DNA,” as used herein, refers to DNA of bacterial origin that does not encode a known antigen. See, for example, Hacker, G., et al., Immunology 105:245-251 (2002); Siders, W. F., Mol. Ther. 6:519-527 (2002); and Klinman, D. M., et al., Proc. Natl. Acad. WSci. USA 93:2879-2883 (1996). Noncoding bacterial DNA polynucleotides can be in linear, circular (e.g., a plasmid), or branched form; and in double-stranded or single-stranded form. Bacterial double-stranded DNA plasmids are preferred for use with the saponin derivatives of the invention.

CpG oligonucleotides can also be used with saponin derivatives of the invention to stimulate a non-specific innate immune response in an animal. The term “CpG oligonucleotide” refers to DNA polynucleotides of about 20 to about 25 nucleotides or less, which contain one or more CpG dinucleotide motifs. CpG oligonucleotides can be single-stranded or double-stranded. Double-stranded DNA CpG oligonucleotides of about 20 base pairs are preferred.

In some aspects of the present invention, the saponin derivatives described herein are administered to an animal in conjunction with a bacterial DNA polynucleotide or a CpG oligonucleotide. The saponin derivatives can associate with the polynucleotides or oligonucleotides (via salt linkages) to form complexes that are fairly stable under physiological conditions. These complexes should be reversible and able to dissociate in the presence of pH changes, or some agents, such as certain proteins or salts, to yield free polynucleotide or oligonucleotide.

Bacterial DNA/saponin derivative complexes and CpG oligonucleotide/saponin derivative complexes may also be administered with an antigen polypeptide or with a coding DNA or RNA vaccine, as described below, to stimulate or generate a specific immunity in an animal. The polypeptide antigen or DNA or RNA vaccine is preferably administered in combination with the bacterial DNA/saponin derivative complexes or in combination with the CpG oligonucleotide/saponin derivative complexes. Thus, for example, in some aspects of the invention, the polypeptide antigen is included in, or forms a part of, the bacterial DNA/saponin derivative complex that is administered to an animal.

The saponin derivatives of the present invention can also be utilized to enhance the immune response of an animal against specific antigens produced by the use of nucleic acid vaccines. Typical vaccines using this approach are viral vaccines, such as influenza, herpes, cytomegalovirus, HIV-1, HTLV-1, FIV, cancer vaccines, and parasitic vaccines. DNA vaccines are also currently being developed for prevention and treatment of a number of infectious diseases. Boyer, J., et al., Nat. Med. 3:526-532 (1997); reviewed in Spier, R., Vaccine 14:1285-1288 (1996).

In a DNA or RNA vaccine, a polynucleotide operatively coding for an immunogenic polypeptide in a pharmaceutically acceptable administrable carrier is administered to the cells of an animal suffering from cancer or pathogenic infection, wherein the polynucleotide is incorporated into the cells and an amount of an immunogenic polypeptide is produced capable of stimulating a preventive or therapeutically effective immune response.

The polynucleotide material delivered to the cells can take any number of forms. It may contain the entire sequence or only a fragment of an immunogenic polypeptide gene. It may also contain sequences coding for other polypeptide sequences. It may additionally contain elements involved in regulating gene expression (e.g., promoter, enhancer, 5′ or 3′ UTRs, transcription terminators, and the like). The polynucleotide may also comprise an immunostimulatory sequence that would enhance the immunogenicity of a given gene product, and/or it may comprise sequences that would enhance the delivery of the polynucleotide, such as by increasing cellular and/or nuclear uptake. Techniques for obtaining expression of exogenous DNA or RNA sequences in a host are known. See, for example, Korman et al., Proc. Nat. Acad. Sci. (USA) 84:2150-2154 (1987), which is hereby incorporated by reference.

The polynucleotide material delivered to the cells can also be antisense DNA or antisense RNA. Thus, in the present invention, the saponin derivatives described herein can be utilized to deliver antisense DNA or RNA into target cells. Cell targeting depends on the sugars attached to the saponin aglycone core. Thus, it is possible to modify or replace one or more oligosaccharide chains in the saponin derivatives chosen for use with the antisense DNA or RNA with other sugar residues that will target the saponin derivative/antisense polynucleotide complex to particular types of cells.

In a preferred aspect of the present invention, the saponin derivatives described herein are administered to an animal in conjunction with a DNA or RNA vaccine comprising a polynucleotide, i.e., DNA or RNA, that encodes an antigen. The saponin derivatives associate with the polynucleotide and facilitate targeting of the polynucleotide to APCs of the animal, such that the polynucleotide is incorporated into the cells of the animal, a therapeutically effective amount of the encoded antigen is produced, and an effective immune response is produced in the animal.

The saponin derivatives administered with the nucleic acid vaccine have the capacity to form complexes with the DNA or RNA polynucleotides of the vaccine (via salt linkages) that are fairly stable under physiological conditions. These complexes should be reversible and able to dissociate in the presence of pH changes, or some agents, such as certain proteins or salts, to yield free DNA or RNA. The strength of the association between the DNA/RNA and the saponin derivative may be gauged by adjusting the length of the cationic chain attached to the saponin moiety and the nature and/or density of its basic groups.

The DNA or RNA complexes formed with the saponin derivatives (i.e., the saponin derivative/polynucleotide complex) disclosed here may also interact with i) native saponins, such as those from quillaja, gypsophila or similar ones; ii) semi-synthetic saponin derivatives such as GPI-0100 and similar ones; or iii) synthetic glycosides containing a triterpenoid aglycone linked to one or more carbohydrate chains. The aglycone core may or may not carry an aldehyde or ketone group. These interactions occur between the saponin moieties of the present invention and the natural or semi-synthetic saponin derivatives to form mixed micelles or similar aggregates in the presence or absence of DNA or RNA. These micelles or aggregates should also occur in the presence of non-ionic detergents, such as polyoxyethylene fatty acid esters, polyoxyethylene sorbitan fatty acid esters, and others, forming mixed micelles containing the non-ionic detergent. The natural glycosides or saponins, their semi-synthetic derivatives and synthetic products capable of interacting with the glycoside or saponin moieties of the present invention, should preferentially have an aldehyde or ketone group to provide a co-stimulatory signal to an APC, and a lipophilic side chain capable of interacting with the cell membrane to facilitate the delivery of the nucleic acid to the cytosol.

The DNA or RNA complex formed with the modified saponins of the present invention should bind to cell receptors for carbohydrates, preferentially on APCs, by the saponins' carbohydrate residues. Alternatively, after forming a saponin derivative/polynucleotide complex, the modified saponins of the present invention would associate with either natural, semi-synthetic or synthetic derivatives of saponins, preferably derivative of triterpenoid saponins, preferentially carrying an aldehyde, to form micelles or similarly aggregated structures. These aggregates would then bind to the cell-surface receptors for the saponins' carbohydrate residues, mediate the delivery of DNA of RNA to the cell's cytosol compartment, and if they contain an aldehyde group, co-stimulate the T-cells. The presence of a co-stimulatory signal like the aldehyde group, may help avoid the problem of “anergy”. This anergy or immune tolerance is caused by the interaction between the T Cell Receptor (TCR) and the APC's MHC-1/peptide complex, but without the concomitant co-stimulation by B7-1. In the present invention, the modified saponin DNA carrier provides such a co-stimulatory signal via aldehyde groups present on the carrier itself or in other glycosides associated with the carrier.

The methods of the invention may be carried out by direct delivery to the mucosal membranes or by direct injection of the saponin derivative/polynucleotide complex into the animal in vivo, or by in vitro transfection of some of the animal cells which are then re-introduced into the animal's body.

Thus, the present invention provides a method of immunizing an animal, wherein a preparation of a saponin derivative/polynucleotide complex is obtained that comprises one or more saponin derivatives of the invention and a polynucleotide construct comprising a polynucleotide coding for an antigenic peptide. The saponin derivative/polynucleotide complex is then introduced into an animal, whereby the polynucleotide construct is incorporated into an APC (a monocyte, a macrophage, a dendritic cell, or another cell), where an antigenic translation product of the polynucleotide is formed, and the product is processed and presented by the cell in the context of the major histocompatibility complex, thereby eliciting an immune response against the antigen. Again, the polynucleotide is DNA or RNA, but preferably mRNA. If the polynucleotide is DNA, the gene for an antigen (“immunogen”) is present on the polynucleotide. If the polynucleotide is mRNA, the mRNA, when translated, produces the antigen.

In an alternative embodiment, the present invention also provides a method of immunizing an animal, wherein one or more cells are removed from an animal and the cells are transfected in vitro with a saponin derivative/polynucleotide complex that comprises one or more saponin derivatives of the invention and a polynucleotide construct comprising a polynucleotide coding for an antigenic peptide. The polynucleotide construct of the complex is incorporated into the cells and an antigenic translation product of the polynucleotide is formed. After transfection, the cells, now expressing the antigen, are reinjected into the animal where the immune system can respond to the (now) endogenous antigen and an immune response against the immunogen is elicited. In this embodiment of the invention, the cells to be transfected with the saponin/polynucleotide complex are preferably lymphoid cells, more preferably APC's, which have been removed from an animal.

If cells from the animal are to be transfected in vitro in practice of the invention, the source of the cells can be peripheral blood cells, which can be rapidly isolated from whole blood to provide a source of cells containing both class I and class II MHC proteins. These cells can be further fractionated into B cells, helper T cells, cytotoxic T cells or macrophage/monocyte cells if desired (APC's). Bone marrow cells can provide a source of less differentiated lymphoid cells. In all cases the cell will be transfected in vitro either with DNA containing a gene for the antigen or by the appropriate capped and polyadenylated mRNA transcribed from that gene or a circular RNA, chemically modified RNA, or an RNA which does not require 5′ capping. The choice of the transfecting nucleotide may depend on the duration of expression desired. For vaccination purposes, a reversible expression of the immunogenic peptide, as occurs on mRNA transfection, is preferred. Transfected cells are injected into the animal and the expressed proteins will be processed and presented to the immune system by the normal cellular pathways.

As used herein, the term “antigen” means a substance that has the ability to induce a specific immune response. For purposes of the present invention, the term “antigen” is used interchangeably with “immunogen”.

Any appropriate antigen which is a candidate for an immune response, whether humoral or cellular, can be used in the invention. In any of the embodiments of the invention, the immunogenic product may be secreted by the cells, or it may be presented by a cell of the animal in the context of the major histocompatibility antigens, thereby eliciting an immune response against the immunogen. The invention may be practiced using non-dividing, differentiated APCs from the vertebrates, such as lymphocytes obtained from a blood sample.

Since the immune systems of all vertebrates operate similarly, the applications described can be implemented in all vertebrate systems, comprising mammalian and avian species, as well as fish. Any vertebrate that may experience the beneficial effects of the compositions and applications of the present invention is within the scope of subjects that may be treated.

The subjects are preferably mammals. The term “mammal” is intended to encompass a singular “mammal” and plural “mammals,” and includes, but is not limited to, primate mammals such as human, apes, monkeys, orangutans, and chimpanzees; canine mammals such as dogs and wolves; feline mammals such as cats, lions, and tigers; equine mammals such as horses, donkeys, deer, zebras, and giraffes; and common domesticated mammals such as cattle, sheep, and pigs. Preferably, the mammal is a human subject.

In a preferred aspect of the invention, the polynucleotide construct of the nucleic acid vaccine to be used with the saponin derivatives of the present invention comprises at least one polynucleotide (e.g., DNA, RNA, ribozyme, phosphorothioate, or other modified nucleic acid) encoding one or more antigens. The polynucleotide can be provided in linear, circular (e.g. plasmid), or branched form; and double-stranded or single-stranded form. The polynucleotide can involve a conventional phosphodiester bond or a non-conventional bond (e.g., an amide bond as in peptide nucleic acid (PNA)). The choice of polynucleotide encoding an antigen will depend on the desired kinetics and duration of expression. When long term delivery of the polynucleotide construct is desired, the preferred polynucleotide is DNA. Alternatively, when short term delivery is desired, the preferred polynucleotide is mRNA. RNA will be rapidly translated into polypeptide, but will be degraded by the target cell more quickly than DNA. In general, because of the greater resistance of circular DNA molecules to nucleases, circular DNA molecules will persist longer than single-stranded polynucleotides, and they will be less likely to cause insertional mutation by integrating into the target genome.

In a preferred embodiment, the polynucleotide sequence encoding one or more antigens is RNA. Most preferably, the RNA is messenger RNA (mRNA). A viral alphavector, a non-infectious vector useful for administering RNA, may be used to introduce RNA into animal cells. Methods for the in vivo introduction of alphaviral vectors to mammalian tissues are described in Altman-Hamamdzic, S., et al., Gene Therapy 4: 815-822 (1997), which is herein incorporated by reference.

In another embodiment of the invention, the polynucleotide sequence encoding one or more antigens is DNA. In a DNA construct, a promoter is preferably operably linked to the polynucleotide encoding an antigen. The promoter may be a cell-specific promoter that directs substantial transcription of the DNA only in predetermined cells. Other transcription control elements, besides a promoter, can be included in the polynucleotide construct to direct cell-specific transcription of the DNA.

An operable linkage is a linkage in which a polynucleotide sequence encoding an antigen is connected to one or more regulatory sequence in such a way as to place expression of the antigen sequence under the influence or control of the regulatory sequence(s). Two DNA sequences (such as a coding sequence and a promoter region sequence linked to the 5′ end of the coding sequence) are operably linked if induction of promoter function results in the transcription of mRNA encoding the desired polypeptide and if the nature of the linkage between the two DNA sequences does not (1) result in the introduction of a frame-shift mutation, (2) interfere with the ability of the expression regulatory sequences to direct the expression of the polypeptide, antisense RNA, or (3) interfere with the ability of the DNA template to be transcribed. Thus, a promoter region would be operably linked to a DNA sequence if the promoter was capable of effecting transcription of that DNA sequence.

Preferably, the polynucleotide construct is a circular or linearized plasmid containing non-infectious, nonintegrating nucleotide sequence. A linearized plasmid is a plasmid that was previously circular but has been linearized, for example, by digestion with a restriction endonuclease. The polynucleotide sequence encoding an antigen may comprise a sequence which directs the secretion of the antigenic polypeptide.

“Noninfectious” means that the polynucleotide construct does not infect mammalian cells. Thus, the polynucleotide construct can contain functional sequences from non-mammalian (e.g., viral or bacterial) species, but the construct does not contain functional non-mammalian nucleotide sequences that facilitate infection of the construct into mammalian cells.

“Nonintegrating” means that the polynucleotide construct does not integrate into the genome of mammalian cells. The construct can be a non-replicating DNA sequence, or specific replicating sequences genetically engineered to lack the ability to integrate into the genome. The polynucleotide construct does not contain functional sequences that facilitate integration of the antigen-encoding polynucleotide sequence into the genome of mammalian cells.

The polynucleotide construct is assembled out of components where different selectable genes, origins, promoters, introns, 5′ untranslated (UT) sequence, terminators, polyadenylation signals, 3′ UT sequence, and leader peptides, etc. are put together to make the desired vector. The precise nature of the regulatory regions needed for gene expression can vary between species or cell types, but shall in general include, as necessary, 5′ non-transcribing and 5′ non-translating (non-coding) sequences involved with initiation of transcription and translation respectively, such as the TATA box, capping sequence, CAAT sequence, and the like, with those elements necessary for the promoter sequence being provided by the promoters of the invention. Such transcriptional control sequences can also include enhancer sequences or upstream activator sequences, as desired.

The polynucleotide construct can be an expression vector. A typical mammalian expression vector contains the promoter element, which mediates the initiation of transcription of m-RNA, the polypeptide coding sequence, and signals required for the termination of transcription and polyadenylation of the transcript, as well as additional elements that include enhancers, Kozak sequences and intervening sequences flanked by donor and acceptor sites for RNA splicing. Suitable expression vectors for use in practicing the present invention include, for example, vectors such as PSVL and PMSG (Pharmacia, Uppsala, Sweden), pRSVcat (ATCC 37152), pSV2dhfr (ATCC 37146), pBC12MI (ATCC 67109), VR1012, VR1055, and pcDNA3 (Invitrogen, San Diego, Calif.). All forms of DNA, whether replicating or non-replicating, which do not become integrated into the genome, and which are expressible, can be used in the methods contemplated by the invention.

The vector containing the DNA sequence (or the corresponding RNA sequence) which can be used in accordance with the invention can be a eukaryotic expression vector. Techniques for obtaining expression of exogenous DNA or RNA sequences in a host are known. See, for example, Korman et al., Proc. Nat. Acad. Sci. (USA) 84:2150-2154 (1987), which is herein incorporated by reference.

The present invention also encompasses the use of DNA coding for a polypeptide and for a polymerase for transcribing the DNA, and wherein the DNA includes recognition sites for the polymerase. The initial quantity of polymerase is provided by including mRNA coding therefor in the preparation, which mRNA is translated by the cell. The mRNA preferably is provided with means for retarding its degradation in the cell. This can include capping the mRNA, circularizing the mRNA, or chemically blocking the 5′ end of the mRNA. The DNA used in the invention may be in the form of linear DNA or may be a plasmid. Episomal DNA is also contemplated. One preferred polymerase is phage T7 RNA polymerase and a preferred recognition site is a T7 RNA polymerase promoter.

For the methods of the present invention, a single polynucleotide construct containing more than one polynucleotide sequence encoding one or more molecules may be used according to the invention. Alternatively, more than one polynucleotide construct each containing polynucleotide sequences encoding one or more molecules may be used as well.

When the single polynucleotide construct containing more than one polynucleotide encoding a polypeptide is DNA, preferably, each polynucleotide encoding a polypeptide will be operably linked to a separate promoter. Alternatively, the polynucleotides encoding polypeptides may be operably linked to the same promoter in order to form a polycistronic transcription unit wherein each sequence encoding a polypeptide is separated by translational stop and start signals. Transcription termination is also shared by these sequences. While both DNA coding sequences are controlled by the same transcriptional promoter, so that a fused message (mRNA) is formed, they are separated by a translational stop signal for the first and start signal for the second, so that two independent polypeptides result. Methods of making such constructs are disclosed in U.S. Pat. Nos. 4,713,339, and 4,965,196, which are herein incorporated by reference.

When the single polynucleotide construct containing more than one polynucleotide encoding a polypeptide is RNA, preferably, there will be separate translational start and stop signals for each polypeptide-encoding sequence in order to produce two or more separate polypeptides.

In the present invention, the polynucleotide construct is complexed with one or more saponin derivatives of the invention by ionic interaction. Generally, the complex then contacts the cell membrane and is transfected into the cell, in a fashion analogous to “lipofection,” a highly efficient transfection procedure, in which DNA or RNA is complexed with one or more cationic lipids for transfection into a cell. See Felgner et al., Proc. Natl. Acad. Sci. USA 84:7413-7417, (Nov. 1987); and Felgner et al., Nature 337:387-388 (1989).

In a formulation for preparing the saponin derivative/polynucleotide complexes of the invention, the saponin derivatives can be present at a concentration of between about 0.1 mole % and about 100 mole %, preferably about 5 mole % and 100 mole %, and most preferably between about 20 mole % and 100 mole %, relative to other compounds present in the formulation.

In preparing the saponin derivative/polynucleotide complexes of the invention, the polynucleotide construct can be solubilized in a buffer prior to mixing with one or more saponin derivatives. Suitable buffers include, for example, phosphate buffered saline (PBS), normal saline, Tris buffer, and sodium phosphate vehicle (100-150 mM preferred). Insoluble polynucleotides can be solubilized in a weak acid or base, and then diluted to the desired volume with a neutral buffer such as PBS. The pH of the buffer is suitably adjusted, and moreover, a pharmaceutically acceptable additive can be used in the buffer to provide an appropriate osmolarity.

The cationic saponin derivatives of the invention are present in solution as either monomers or as micelles, depending on the concentration of the saponin derivative, and on the ionic strength and pH of the solution. Because of their cationic nature, these derivatives tend to have critical micellar concentration values higher than those of the non-ionic derivatives such as alkylamide saponin derivatives. Cationic saponin derivatives can be prepared in water, isotonic solutions of 5% mannitol or sorbitol, or low ionic strength buffers, and mixed with the polynucleotide dissolved in a buffer solution containing 0.15 M NaCl, mannitol or sorbitol, to form a saponin derivative/polynucleotide complex. Cationic saponin derivatives can also be used in conjunction with alkylamide saponin derivatives by mixing them together prior to adding the polynucleotide. Alternatively, the alkylamide saponin derivatives can be added to the cationic saponin derivative/polynucleotide complex to form a mixed micelles system containing the polynucleotide.

Cationic saponin derivatives of the invention with lipophilic chains containing 18 or more carbon atoms may form vesicles that are heterogeneous in size, particularly if they are mixed with alkylamide saponin derivatives having lipid chains containing 18 or more carbon atoms. Therefore, according to a preferred method, such cationic saponin derivatives are prepared by dissolution in a chloroform-methanol solvent mixture, and the resulting cationic saponin derivative/chloroform-methanol mixture is evaporated to dryness as a film on the inner surface of a glass vessel. On suspension in an aqueous solvent, these cationic saponin derivatives assemble themselves into vesicles. These vesicles are reduced to a selected mean diameter by means of a freeze-thaw procedure. Vesicles of uniform size can be formed prior to drug delivery according to methods for vesicle production known to those in the art; for example, the sonication of a lipid solution as described by Felgner et al., Proc. Natl. Acad. Sci. USA 84:7413-7417 (1987) and U.S. Pat. No. 5,264,618, which are herein incorporated by reference. Once the vesicles have been formed by suspension in aqueous solvent, they are added with stirring to the polynucleotide solution, to entrap the polynucleotide within the vesicles or to form a complex of cationic saponin and polynucleotide.

The saponin derivative/polynucleotide complexes of the invention may be delivered to any tissue, including, but not limited to, muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, or connective tissue. Preferably, the construct is delivered to muscle. The muscle may be skeletal or cardiac. Most preferably, the construct is delivered to skeletal muscle.

Preferably, the saponin derivative/polynucleotide complex is delivered to the interstitial space of tissues. “Interstitial space” comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels.

The saponin derivative/polynucleotide complexes can be administered by any suitable route of administration, including intramuscularly, subcutaneously, intravenously, transdermally, intranasally, by inhalation, or transmucosally (i.e., across a mucous membrane, for example by direct application to mucosal surfaces either as drops or as aerosols). Similarly, the pharmaceutical composition of the present invention can by administered by any suitable route, including intramuscularly, into a cavity (e.g., intraperitoneally), subcutaneously, intravenously, transdermally, intranasally, by inhalation, or transmucosally (i.e., across a mucous membrane, for example by direct application to mucosal surfaces either as drops or as aerosols).

Any mode of administration can be used. This includes needle injection, catheter infusion, biolistic injectors, particle accelerators (i.e., “gene guns”, pneumatic “needleless” injectors, e.g., Med-E-Jet (Vahlsing, H. et al., J. Immunol. Methods 171:11-22 (1994)), Pigjet (Schrijver, R. et al., Vaccine 15: 1908-1916 (1997)), Biojector (Davis, H. et al., Vaccine 12:1503-1509 (1994); Gramzinski, R. et al., Mol. Med. 4: 109-118 (1998))), gelfoam sponge depots, other commercially available depot materials, osmotic pumps (e.g., Alza minipumps), oral or suppositorial solid (tablet or pill) pharmaceutical formulations, and decanting or topical applications during surgery. The preferred mode is injection.

Determining an effective amount of substance to be delivered can depend upon a number of factors including, for example, the chemical structure and biological activity of the substance, the age and weight of the animal, the precise condition requiring treatment and its severity, and the route of administration. The precise amount, number of doses, and timing of doses will be determined by the attending physician or veterinarian.

In humans, between 0.5 mg to 40 mg saponin derivative/polynucleotide complex is delivered. Preferably, between 1 mg and 10 mg saponin derivative/polynucleotide complex is delivered, with the polynucleotide comprising 10-15% w/w of the complex.

In certain embodiments, the saponin derivative/polynucleotide complexes are administered as a pharmaceutical composition. The pharmaceutical composition can be formulated according to known methods for preparing pharmaceutical compositions, whereby the substance to be delivered is combined with a pharmaceutically acceptable carrier vehicle. Suitable vehicles and their preparation are described, for example, in Remington 's Pharmaceutical Sciences, 16^{th} Edition, A. Osol, Ed., Mack Publishing Co., Easton, Pa. (1980), and Remington's Pharmaceutical Sciences, 19^{th} Edition, A. R. Gennaro, Ed., Mack Publishing Co., Easton, Pa. (1995).

The pharmaceutical composition can be in the form of an emulsion, gel, solution, suspension, or other form known in the art. In addition, the pharmaceutical composition can also contain pharmaceutically acceptable additives including, for example, diluents, binders, stabilizers, and preservatives. Administration of pharmaceutically acceptable salts of the saponin derivative/polynucleotide complexes described herein is preferred. Such salts can be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like.

For aqueous pharmaceutical compositions used in vivo, sterile pyrogen-free water is preferred. Such formulations will contain an effective amount of the substance together with a suitable amount of vehicle in order to prepare pharmaceutically acceptable compositions suitable for administration to a human or animal.

A pharmaceutical composition can be in solution form, or alternatively, in lyophilized form for reconstitution with a suitable vehicle, such as sterile, pyrogen-free water. Both liquid and lyophilized forms will comprise one or more agents, preferably buffers, in amounts necessary to suitably adjust the pH of the injected solution.

The container in which the pharmaceutical formulation is packaged prior to use can comprise a hermetically sealed container enclosing an amount of the lyophilized formulation or a solution containing the formulation suitable for a pharmaceutically effective dose thereof, or multiples of an effective dose. The pharmaceutical formulation is packaged in a sterile container, and the hermetically sealed container is designed to preserve sterility of the pharmaceutical formulation until use. Optionally, the container can be associated with administration means and or instruction for use.

In certain embodiments of the invention, the saponin derivative/polynucleotide complexes are delivered with additional antiviral agents. Antiviral agents include, but are not limited to, protease inhibitors, nucleoside RT inhibitors, non-nucleoside RT inhibitors, fusion/binding inhibitors, and pyrophosphate analogue RT inhibitors.

Typical vaccines using the saponin derivatives of the invention include viral vaccines, such as influenza, herpes, cytomegalovirus, HIV-1, HTLV-1, FIV, cancer vaccines, and parasitic vaccines.

Applications of the present invention include vaccination against viruses in which antibodies are known to be required or to enhanced viral infection. There are two strategies that can be applied here. One can specifically target the cellular pathway during immunization thus eliminating the enhancing antibodies. Alternatively one can vaccinate with the gene for a truncated antigen which eliminate the humoral epitomes which enhance infectivity. The use of DNA or mRNA vaccine therapy could similarly provide a means to provoke an effective cytotoxic T-cell response to weakly antigenic tumors.

A second application is that this approach provides a method to treat latent viral infections. Several viruses (for example, Hepatitis B, HIV and members of the Herpes virus group) can establish latent infections in which the virus is maintained intracellularly in an inactive or partially active form. There are few ways of treating such an infections. However, by inducing a cytolytic immunity against a latent viral protein, the latently infected cells will be targeted and eliminated.

A related application of this approach is to the treatment of chronic pathogen infections. There are numerous examples of pathogens which replicate slowly and spread directly from cell to cell. These infections are chronic, in some cases lasting years or decades. Examples of these are the slow viruses (e.g. Visna), the Scrapie agent and HIV. One can eliminate the infected cells by inducing an cellular response to proteins of the pathogen.

Finally, this approach may also be applicable to the treatment of malignant disease. Vaccination to mount a cellular immune response to a protein specific to the malignant state, be it an activated oncogene, a fetal antigen or an activation marker, will result in the elimination of these cells.

The use of saponin derivatives of the invention with DNA/mRNA vaccines could in this way greatly enhance the immunogenicity of certain viral proteins, and cancer-specific antigens, that normally elicit a poor immune response. The mRNA vaccine technique should be applicable to the induction of cytotoxic T cell immunity against poorly immunogenic viral proteins from the Herpes viruses, non-A, non-B hepatitis, and HIV, and it would avoid the hazards and difficulties associated with in vitro propagation of these viruses. For cell surface antigens, such as viral coat proteins (e.g., HIV gp120), the antigen would be expressed on the surface of the target cell in the context of the major histocompatibility complex (MHC), which would be expected to result in a more appropriate, vigorous and realistic immune response.

Finally, in the case of the DNA/mRNA vaccines, the protein antigen is never exposed directly to serum antibody, but is always produced by the transfected cells themselves following translation of the mRNA. Hence, anaphylaxis should not be a problem. Thus, the present invention permits the patient to be immunized repeatedly without the fear of allergic reactions. The use of the DNA/mRNA vaccines with the saponin derivatives of the present invention makes such immunization possible.

Parenteral or transmucosal administration to an animal of coding or noncoding bacterial DNA complexed with cationic saponin derivatives stimulates a non-specific innate immunity with the production of cytokines and natural killer (NK) cells with anti-tumor activity and effective in the treatment of cancer. Formulations of bacterial DNA:cationic saponin derivatives in combination with an antigen would stimulate a specific humoral and T-cell immune response against such antigen and useful in the development of preventive and therapeutic vaccines. Bacterial DNA:cationic saponin derivatives can also be administered in combination with other immune modulatory compounds such as QS-21, GPI-0100, immune stimulatory polysaccharides and their derivatives, monophosphoryl lipid A (MPL), muramyl dipeptides (MDP), alum, and others, to provide a synergistic response.

Having now generally described the invention, the same will become more readily understood by reference to the following specific examples which are included herein for purposes of illustration only and are not intended to be limiting unless otherwise specified.

### EXAMPLES

### Example 1

### Preparation of Group 1 Saponin Derivatives

Schemes 1a-1c illustrate the syntheses of compounds a-c, respectively, as described below.

a) 3-dimethylamino-1-propylamine-saponin derivative. Hydrolyzed or deacylated (DS) quillaja saponin (compound I in Scheme 1a), gypsophylla saponin or a similar one (2.5 g, ˜1.5 mmol) was dissolved in dry

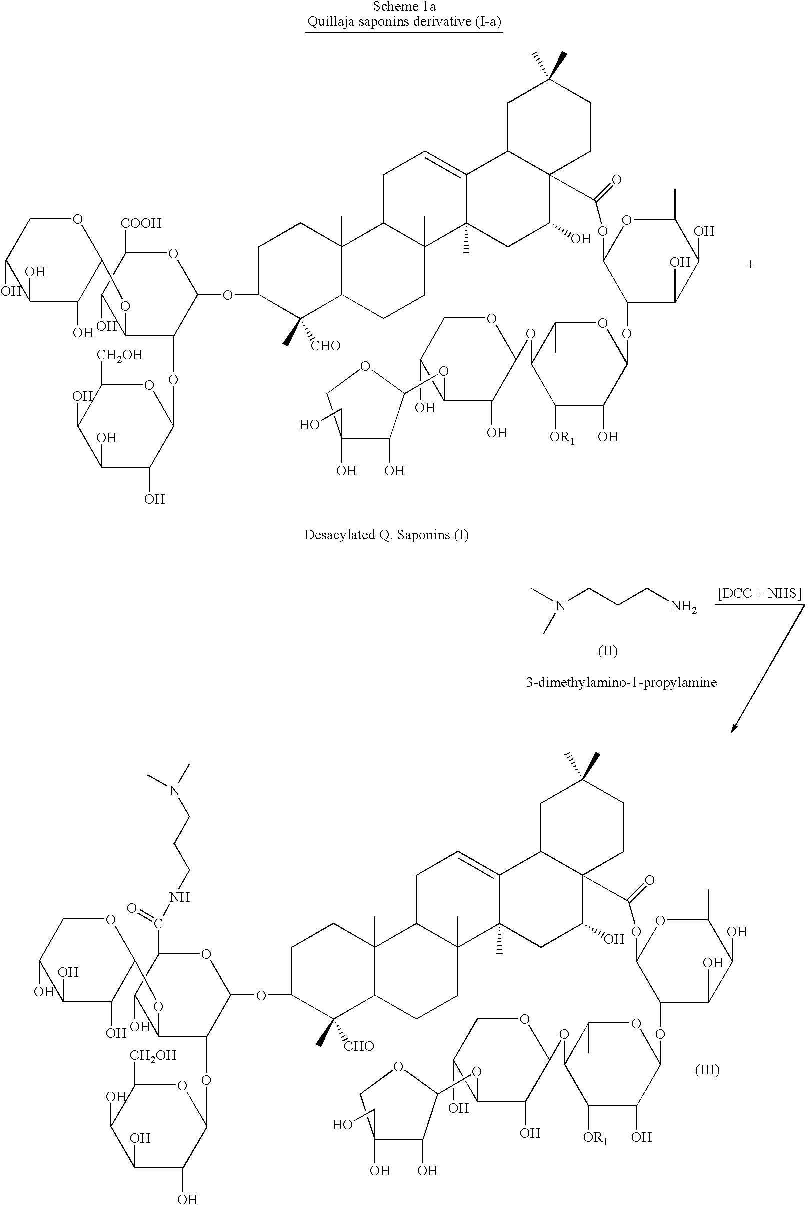

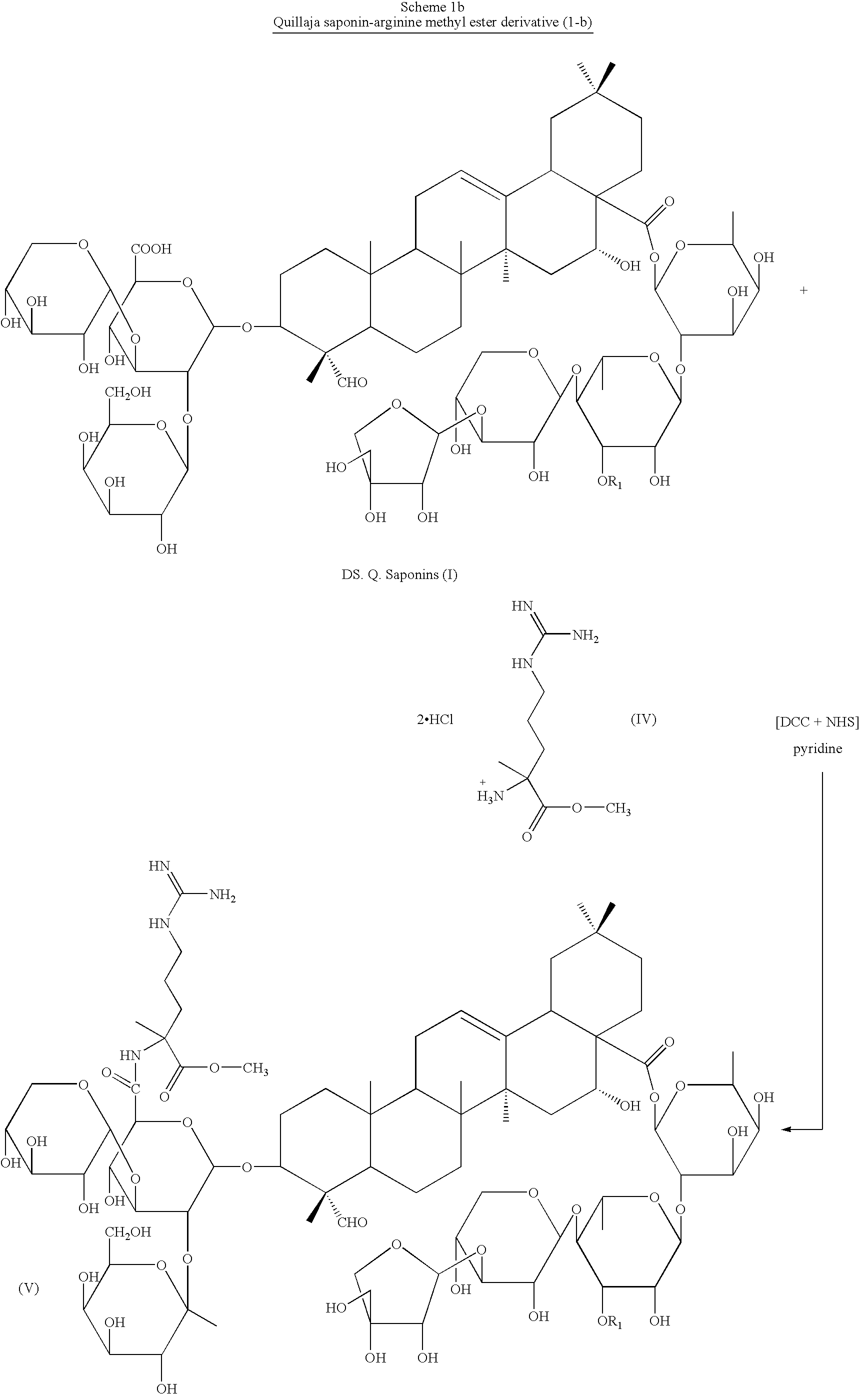

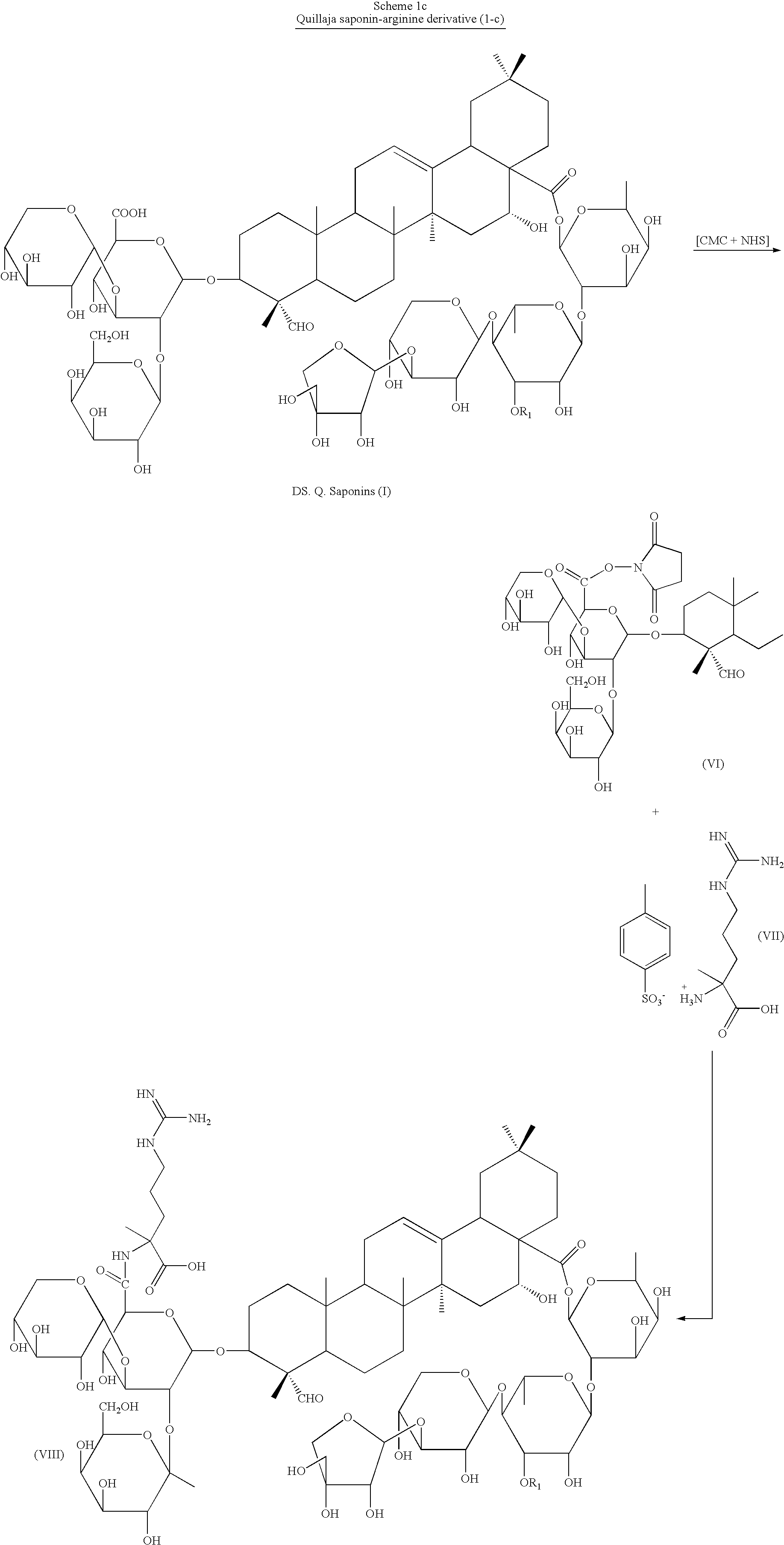

pyridine (25 mL), and 1,3-dicyclohexyl carbodiimide (DCC) (0.93 g, 4.5 mmol), and N-hydroxy succinimide (NIHS) (0.52 g, 4.5 mmol), each dissolved in 12.5 mL of pyridine, were added with vigorous stirring. Subsequently, 3-dimethylamino-1-propylamine (0.46 g, 4.5 mmol) (II) dissolved in pyridine (10 mL) was added dropwise and with stirring over a period of 30 min. The reaction was allowed to proceed with stirring for 2 days. Glacial acetic acid (0.2 mL) was added to the reaction, and the mixture was stirred overnight at room temperature. The resulting suspension was added to distilled water (200 mL) and stirred overnight. Precipitated material (mostly N,N′-dicyclohexyl urea) was collected by filtration. The filtrate was evaporated on a rotary evaporator to remove the pyridine.

The resulting syrup containing the derivatized saponin was diluted with water, put in dialysis bags (M.W. cut off ˜3,000), and dialyzed against several changes of an aqueous solution of 40 mmolar acetic acid for four days. The resultant precipitate was filtered and the clear solution was shelled and lyophilized to get the powdered saponin 3-dimethylamino-1-propylamide derivative (III). The preparation can be further purified by reverse phase chromatography on RP-18 or one similar.

b) Arginine methyl ester-saponin derivative. To 2.5 gm (˜1.5 mmoles) of DS quillaja saponins (I) dissolved in 25 mL of pyridine, was added 4.5 mmoles (0.93 g) of dicyclohexylcarbodiimide (DCC) and 4.5 mmoles (0.52 g) of N-hydroxysuccinimide (NHS), each dissolved in 12.5 mL of pyridine each. To the reaction mixture 0.8 g of L-arginine methyl ester dihydrochloride (IV) (˜3.0 mmol) dissolved in methanol (10 mL) was added drop wise with stirring over a period of 2 hours. The reaction was then allowed to proceed with stirring for 2 days. Glacial acetic acid (0.2 mL) was added to the reaction, and the mixture was stirred overnight at room temperature. The resulting suspension was added to distilled water (250 mL) and stirred overnight. Precipitated material (mostly N,N′-dicyclohexyl urea) was removed by filtration. The clear filtrate was evaporated on a rotary evaporator to remove the pyridine. The resulting syrup containing the derivatized saponin was diluted with water put in dialysis bags (M.W. cutoff ˜3,000) and dialyzed against several changes of 40 mmolar acetic acid for 3 days. Continue dialysis against several changes of water, filter the dialyzed solution, and lyophilize to obtain the dry arginine methyl ester-saponin (V).

c) Arginine-saponin derivative using water-soluble carbodiimide. Two (2) g of purified desacylsaponins (I) (˜1.20 mmoles) were dissolved in 20 ml of pyridine at room temperature, and added with mixing about 1.50 mnoles of CMC (0.64 g of CMC and 2.0 mmoles of N-hydroxysuccimide (0.23 g of NHS). If needed, more pyridine may be added to dissolve the reactants. The reaction was allowed to proceed with mixing overnight at room temperature under anhydrous conditions. Most of the pyridine was removed by rotary evaporation at room temperature. Added to the syrupy residue was 250 ml, of isopropanol to precipitate the saponin intermediate (VI) and collect it by filtration. The ppt. was washed on filter paper with isopropanol to remove the excess of CMC, CMC urea and NHS. The intermediate (VI) (˜1.20 mmoles) was dissolved in ˜25 mL of 50% pyridine and added to 0.45 g (˜2.5 mmole) of L-arginine (2-amino-5-guanidinopentanoic acid) (VII) dissolved in 15 ml of water plus p-toluenesulfonic acid adjusted to pH ˜7. Reacted with mixing for 24 hours at room temperature yielded the saponin analog with an arginine side chain (VIII). If needed the pH of the reaction can be adjusted to ˜7-8 by the addition of aqueous 4 M p-toluenesulfonic acid. In a rotary evaporator the pyridine was removed from the reaction mixture, the syrupy residue was dissolved in 40 mM acetic acid and dialyzed against several changes of this solution for 2 days to remove free arginine. Dialyze against several changes of water, filter, and lyophilize to obtain the dry arginine-saponin derivative (VIII).

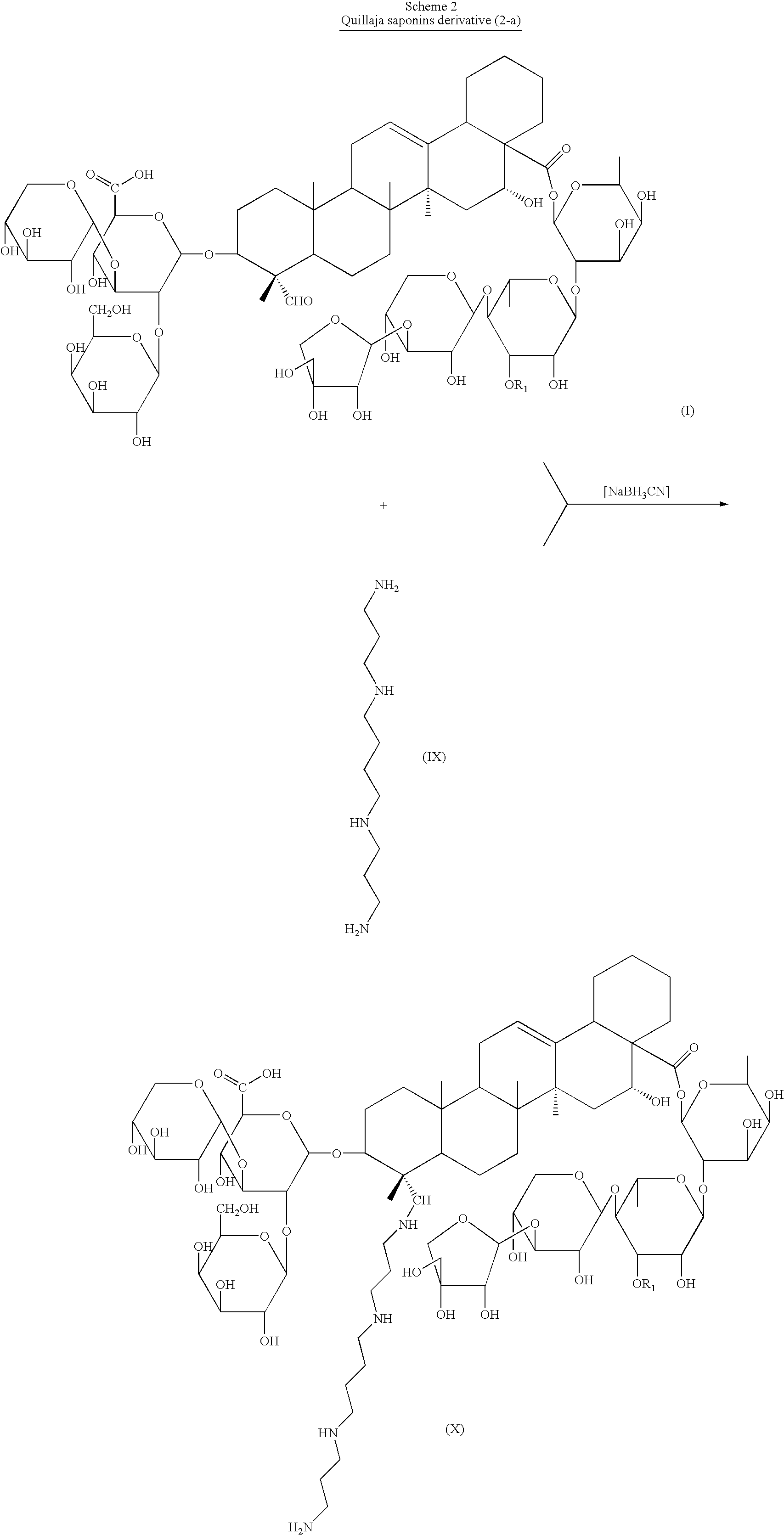

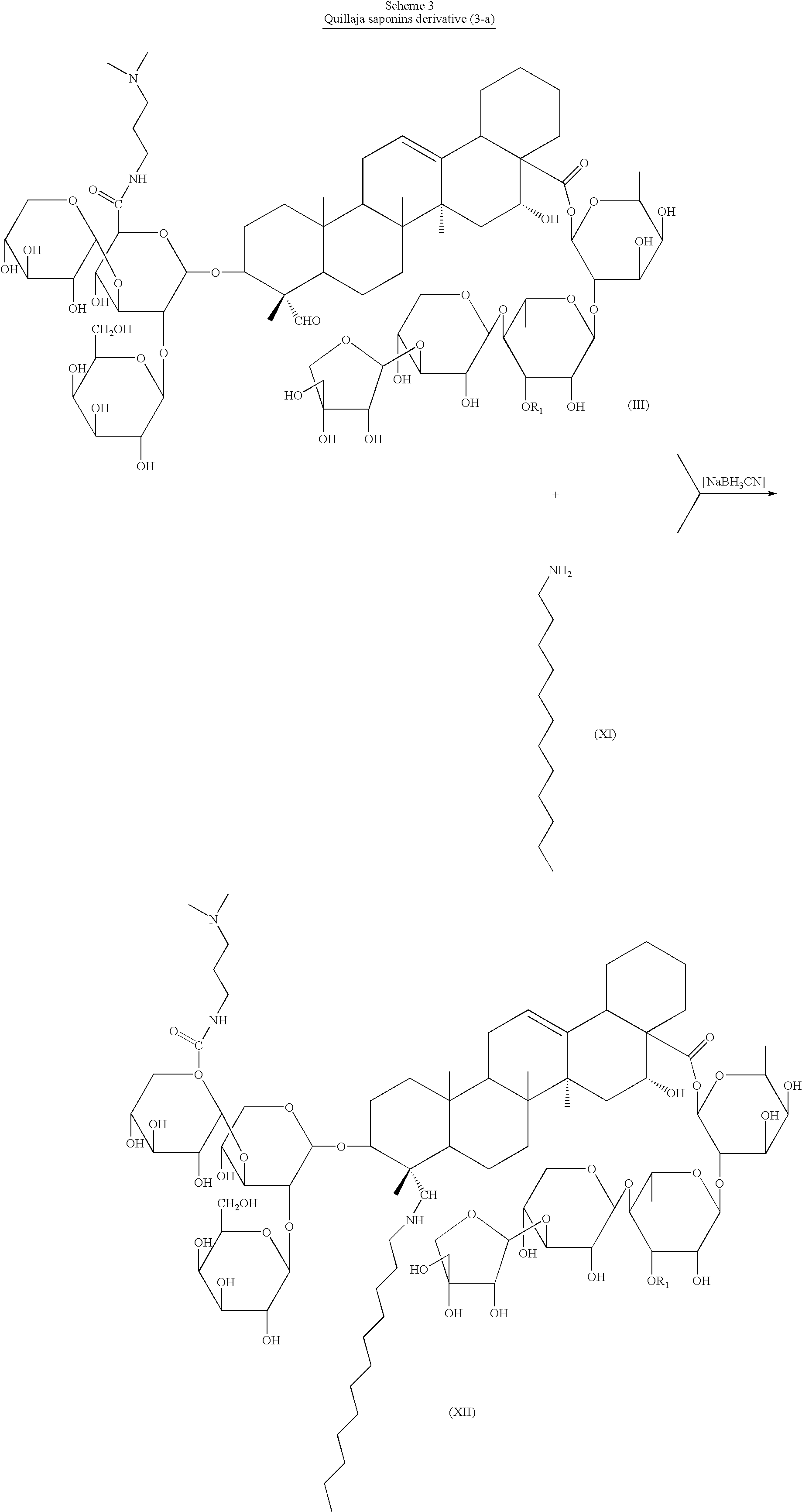

### Example 2

### Preparation of Group 2 Saponin Derivatives

Scheme 2 illustrates the synthesis of compound a, as described below.

a) Saponin-spermine aldehydic derivative. To 1.2 g of spermidine (IX) (6 mmoles) dissolved in 50 mL of aldehyde-free methanol, adjusted to pH ˜9 with acetic acid, and containing 0.12 g of Na cyanoborohydride (˜2 mmoles) over a 4 hour period add dropwise with stirring 2 g of desacylsaponins (I) (˜1.20 mmoles) dissolved in 20 ml of 50% pyridine. The reaction was allowed to proceed for 72 hours to allow the formation of an imine between the spermine primary amines and the triterpenoid aldehyde and its subsequent reduction by Na cyanoborohydride to form a stable secondary amine linkage (X). The reaction mixture was dialyzed against water, followed by dialysis against several changes of 10 mM acetic acid, and lyophilized.

### Example 3

### Preparation of Group 3 Saponin Derivatives

Scheme 3 illustrates the synthesis of compound a, as described below.

a) 3-dimethylamino-1-propylamine-dodecylamine saponin derivative. The 3-dimethylamino-1-propylamine-saponin derivative (III) was prepared as described under 1-a. To 1.1 g of dodecylamine (XI) (6 mmole) dissolved in 50 mL of 50% dimethylformamide, pH ˜8-9, and containing 0.12 g of Na cyanoborohydride (˜2 mmoles), 2 g of derivative (III) (˜1.20 mmoles) dissolved in 20 ml of 50% pyridine were added dropwise with stirring over a 4 hour period. The reaction was allowed to proceed for 48 hours to allow the formation of an imine between a dodecylamine and the triterpenoid aldehyde and its subsequent reduction by Na cyanoborohydride to form a stable secondary amine linkage. The reaction mixture was poured into 1 L of isopropanol to precipitate the 3-dimethylamino-1-propylamine-saponin-dodecylamine derivative (XII). The precipitated material was collected by filtration, washed with isopropanol, dissolved in a minimal volume of 0.1 M acetic acid and dialyzed against several changes of 40 mM acetic acid, followed by dialysis against 10 mM acetic acid. Precipitated material was removed and the clear solution lyophilized.

### Example 4

### Preparation of Group 4 Saponin Derivatives

Scheme 4 illustrates the synthesis of compound a, as described below.

a) Dodecyl amide saponin-spermine aldehydic derivative. To D.S. quillaja saponin, gypsophylla saponin or a similar one (2.5 g, ˜1.5 mmol) dissolved in dry pyridine (25 mL), were added with vigorous stirring 1,3-dicyclohexyl carbodiimide (DCC) (0.93 g, 4.5 mmol), and N-hydroxy succinimide (NHS) (0.52 g, 4.5 mmol), each dissolved in 12.5 mL of pyridine. Subsequently, dodecylamine (0.83 g, 4.5 mmol) dissolved in pyridine (10 mL) was added dropwise and with stirring over a period of 30 min. The reaction was allowed to proceed with stirring for 2 days. Glacial acetic acid (0.2 mL) was added to the reaction, and the mixture was stirred overnight at room temperature. The resulting suspension was added to distilled water (200 mL) and stirred overnight. The precipitated material (mostly N,N′-dicyclohexyl urea) was removed by filtration. The clear filtrate was evaporated on a rotary evaporator to remove the pyridine. The resulting syrup containing the derivatized saponin was diluted with water delivered into dialysis bags (M.W. cut off ˜12,000) and dialyzed against several changes of an aqueous solution of 40 mmolar acetic acid for four days. The resulting precipitate was filtered and the clear solution was shelled and lyophilized to get the dry dodecylamide saponin derivative (XIII). To 1.2 g of spermidine (IX) (6 mmoles) dissolved in 50 mL of aldehyde-free methanol, and containing 0.12 g of Na cyanoborohydride (˜2 mmoles) add dropwise with stirring and over a 6-8 hours period 2 g of dodecylamide saponin (XIII) (˜1.20 mmoles) dissolved in

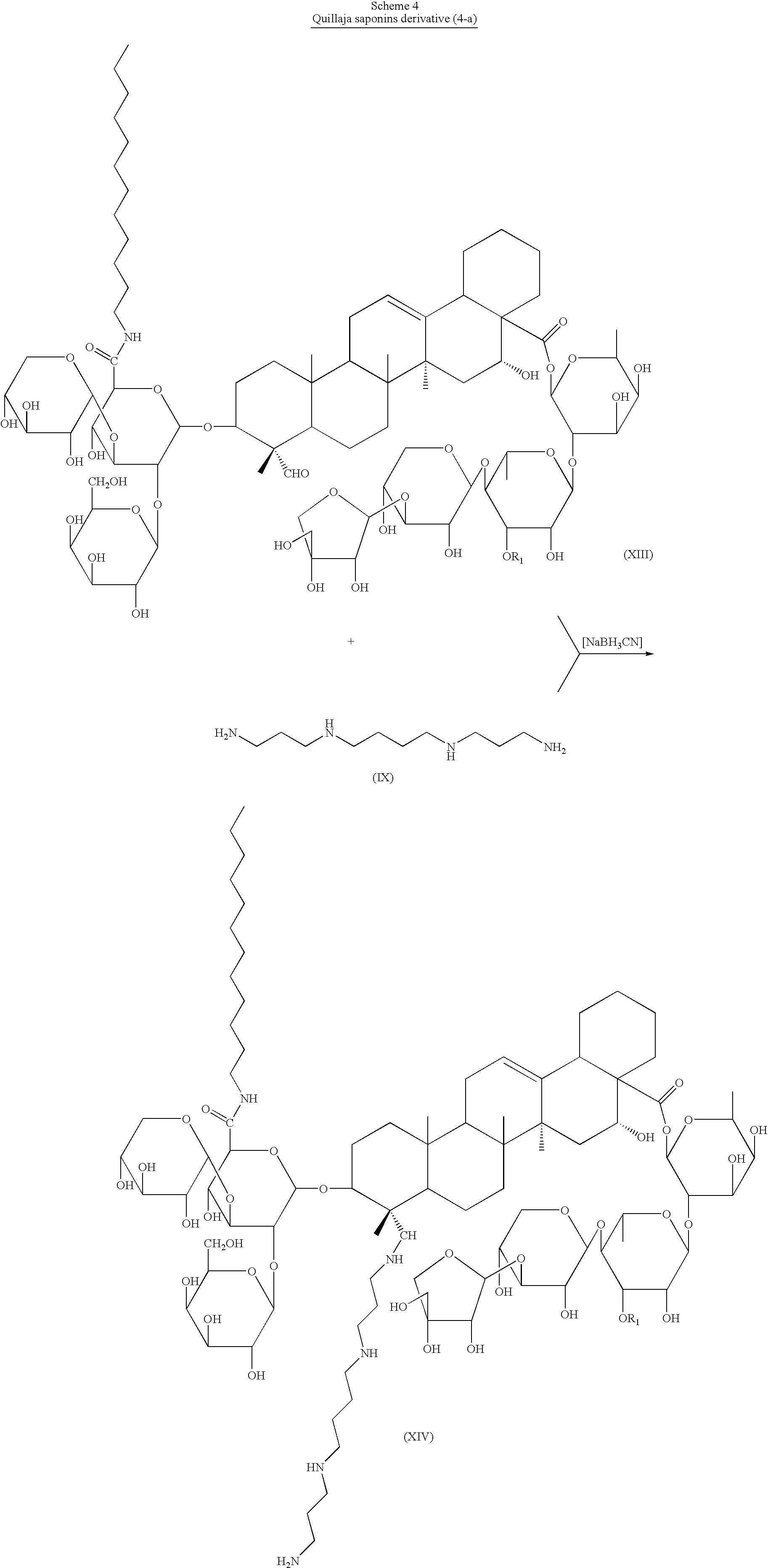

20 ml of aldehyde-free methanol. The reaction was allowed to proceed for 72 hours to allow the formation of an imine between the spermine primary amines and the triterpenoid aldehyde and its subsequent reduction by Na cyanoborohydride to form a stable secondary amine linkage. The dodecyl amide saponin-spermine aldehydic derivative (XIV) was separated from the excess reactants by gel filtration on Sephadex G-15, using water as an eluent. The void volume peak containing the derivative (XIV) was lyophilized. Further purification can be achieved by reverse chromatography on Silica RP-18 using a methanol-water gradient with 50 mM acetic acid, or using ion exchange chromatography using a DEAE matrix and a salt gradient at acid pH. Collect fraction containing the derivative (XIV) and remove the salt by gel filtration on Sephadex G-15 using water as eluent and lyophilize the void volume peak containing (XIV).

### Example 5

### Preparation of Group 5 Saponin Derivatives

Schemes 5a-5c illustrate the syntheses of compounds a-c, respectively, as described below.

a) Histone-dodecylamide saponin derivatives. To 1.10 g (˜10 mmoles a.a., ˜1-2 mmoles NH2)) of histones dissolved in 40 mL of 6 M urea, 0.1 M HEPBS buffer pH 8.0, were added 0.5 mmole (0.9 g) of the dodecylamide saponin derivative (XIII) dissolved in 10 mL of pyridine, and 0.3 g (0.5 mmole) of Na cyanoborohydride dissolved in 5-10 ml pyridine, and allowed to react with stirring for 72 hours at room temperature. The reaction mixture was dialyzed against several changes of water to remove the excess of reactants. To the dialyzed solution, containing some precipitated material, acetic acid was added to re-dissolve the historic derivative (XV). After filtration, the clear solution was lyophilized to recover the derivative (XV). The histone derivative had a degree of substitution ˜0.05 or 1 residue of dodecylamide saponin derivative for every 20 amino acid residues.

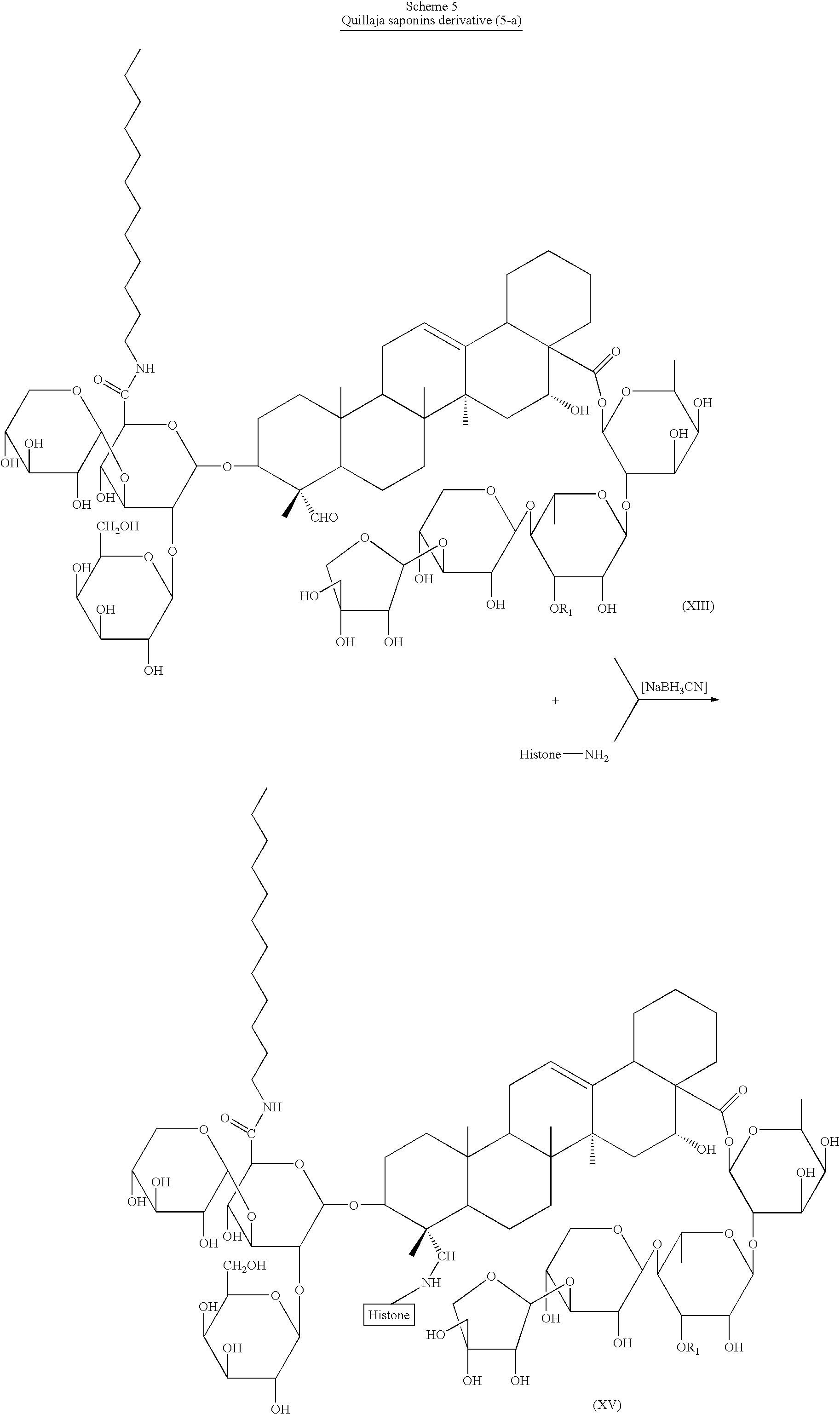

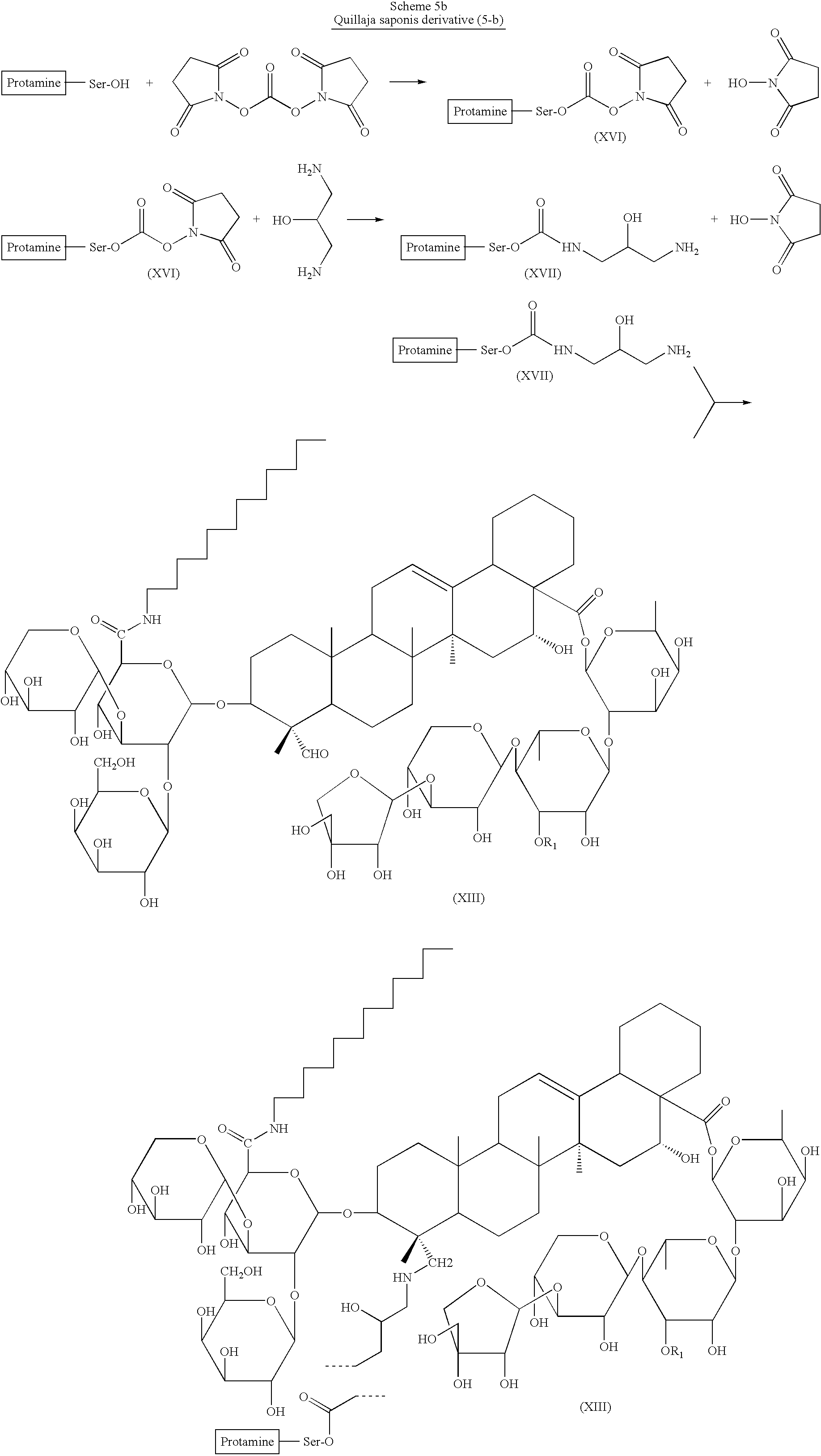

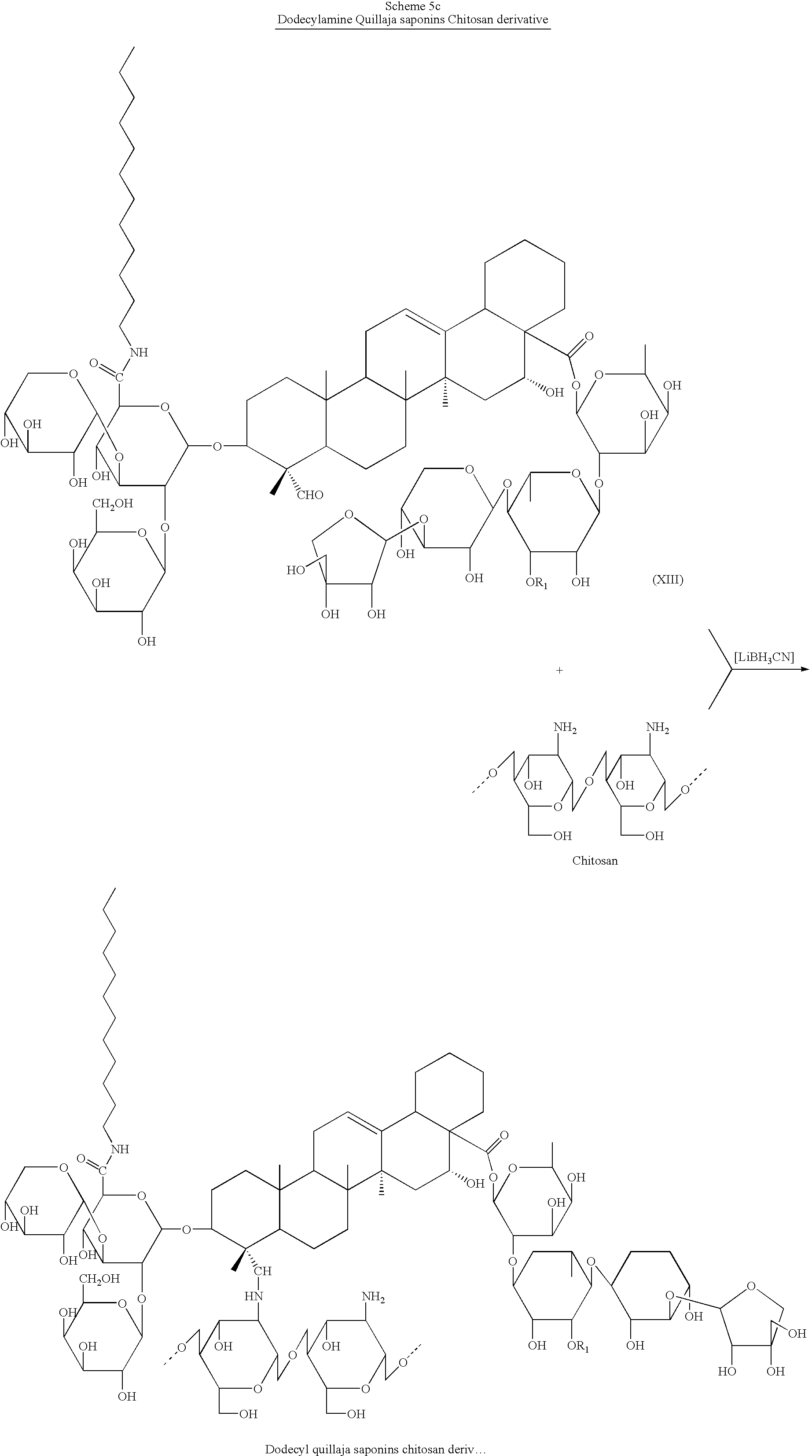

Alternatively, the histone dodecylamide derivative (XV) was separated from the excess reactants by gel filtration of Sephadex G-25 (medium) equilibrated with 20 mM acetic acid. To the reaction enough acetic acid was added in a chemical hood to adjust the concentration to 20 mM acetic acid and stirred for ˜1 hour. The reaction mixture was applied to the Sephadex G-25 column and eluted with 20 mM acetic acid. The void volume was collected and lyqphilized to recover the histone derivative (XV). The histone derivative had a degree of substitution (d.s.) ˜0.05 or 1 residue of dodecylamide saponin derivative for every 20 amino acid residues. The d.s. was determined by estimation of the histones and saponin concentration using the biuret and anthrone reactions for protein and carbohydrate respectively.

b) Protamine-dodecylamide Q. saponin derivatives. To 1 g of protamine, (salmine-free base containing ˜9 mmoles a.a., ˜0.74 mmoles serine), dissolved in 25 mL of anhydrous dimethylsulfoxide, were added with stirring 0.12 g (0.45 mmoles) of N,N′-disuccinimidyl carbonate dissolved in 2 ml of DMSO. To this mixture was added slowly and with stirring 0.055 g (0.5 mmoles) of 4-dimethylaminopyridine dissolved in 1 mL of dioxane or dimethylformamide and reacted overnight at room temperature to form the succinimidyl carbonate-protamine intermediate (XVI) with a d.s. ˜0.05. To the intermediate (XVI), 0.36 g of 1,3-diamino-2-propanol (a 8× excess over the succinimyl carbonate) dissolved in 2 mL DMSO were added, and the reaction was allowed to continue with stirring for 48 at RT hours to form a protamine-propylamine derivative (XVII). The intermediate (XVII) was precipitated over night by adding the reaction with stirring into 400-500 mL of acetone with 5% glycerol. The precipitate was collected by filtration, and washed by gravity or gentle suction with several volumes of acetone glycerol (95 v/5 v). The collected material was not allowed to get dry. To the precipitated intermediate (XVII) dissolved in 40 mL of freshly prepared 8 M urea solution, 0.1 M HEPBS buffer pH 8.0, were added 1 mmole (1.8 g) of dodecylamide saponin derivative (XIII) dissolved in 10 mL of pyridine, and 0.4 g (0.67 mmole) of Na cyanoborohydride dissolved in 5-10 ml pyridine. The reaction was allowed to continue with stirring for 72 hours at R.T. The reaction mixture was dialyzed against several changes of water, followed by 20 mM acetic acid to remove excess of reactants, filtered and lyophilized to recover the histone derivative (XVIII). The (XVIII) derivative had a d.s. ˜0.05 or 1 residue of dodecylamide saponin derivative for every 20 amino acid residues. Alternatively, the excess of reactants was separated from the protamine-propylamine derivative (XVII) by gel filtration on Sephadex G-25. To the reaction mixture in DMSO enough urea was added to make it ˜6M and apply it to a Sephadex G-25 column equilibrated with 40 mM acetic acid and separate (XVII) from the excess reactants by eluting with 40 mM acetic acid. The void volume containing the protamine derivative was collected, concentrated on a rotary evaporator and lyophilized. The lyophilized material was dissolved in 40 mL of freshly prepared 8 M urea solution, 0.1 M HEPBS buffer pH 8.0, and 1 mmole (1.8 g) of dodecylamide saponin derivative (XIII) dissolved in 10 mL of pyridine, and 0.4 g (0.67 mmole) of Na cyanoborohydride dissolved in 5-10 ml pyridine were added. The reaction was allowed to continue with stirring for 72 hours at R.T. The reaction mixture was dialyzed against several changes of water. Any formed precipitate was re-dissolved by adding to the protamine solution acetic acid to adjust the pH to −7. The solution was filtered and lyophilized to recover the histone derivative (XVIII). The (XVIII) derivative had a degree of substitution ˜0.05 or 1 residue of dodecylamide saponin derivative for every 20 amino acid residues. The d.s. was determined by estimation of the histones and saponin concentrations using the biuret and anthrone reactions for proteins and carbohydrates respectively.

c) Dodecylamine saponin-chitosan derivative. Commercial crab or shrimp chitosan (˜85% deacetylated) is further deacetylated by autoclaving a suspension of chitosan (10% w/v) in 40% (10N) NaOH at 120° C. for 3 hours. After 3 hours the NaOH containing reaction is dissolved with water 10 fold and the chitosan is left to sediment overnight. Discard the supernatant and wash the deacetylated chitosan several times by decantation with 10-20 volumes of water to bring the pH ˜9. Re-suspend the chitosan in ethanol, collect and wash on filter paper, and store over desiccant.

The chitosan molecular weight ˜200,000 to 300,000, is fragmented using hydrogen peroxide (Chang K L B, et al., J. Agric Food Chem 49:4845-51 (2001)). To chitosan dissolved in 2% acetic acid to yield a 1% solution, hydrogen peroxide (30% w/w) is added to yield a final concentration of 1.5% (dilution 1:20) and the reaction mixture is left to react at 50° C. for about 5 hours. After 5 hours, the reaction is cooled in an ice bath. The average molecular weight of the fragmented chitosan should be around 12,000. Add to the reaction Chelex 100 resin, about 0.2 g per 100 mL reaction, to sequester the metal ions and stop the reaction. Remove the resin by filtration and dialyze the reaction mixture using a 12,000 M.W. cut off membrane against several changes of 0.2 M acetic acid to remove the hydrogen peroxide and small M.W. oligosaccharides. Lyophilize the dialyzed material.

To 1 g of fragmented chitosan (6.2 mmoles glcN) dissolved in 0.1 M acetic acid add 1 g (˜0.6 mmoles) of GPI-0100, and bring the pH to ˜5 with LiOH. Add to the reaction mixture 0.1 mmole of Li cyanoborohydride and let react for 72 hours with gentle stirring. The product is precipitated by addition of 10 volumes of ethanol or isopropanol. Wash the precipitated material with ethanol, dissolve it in a minimal volume of 0.1 M acetic acid, adjust the pH to 5 with LiOH, and re-precipitate with 10 vol. of ethanol. Collect and wash with ethanol over filter paper and store over dessicant. The product is analyzed by reverse phase HPLC using a acetonitrile-water gradient at pH˜9. The degree of submission is to be determined colorimetrically from the differential between the amino groups before and after modification using the TNBS reaction.

d) Polyethylenimine quillaja saponin derivative. To 2 g of DS quillaja saponins (I) (1.2 mmoles) dissolved in 50 mL of anhydrous pyridine were added, with vigorous stirring, 0.744 g DCC (3.6 mmoles) and 0.412 g NHS (3.6 mmoles), and the mixture was stirred for 30 minutes. To this mixture, 1.42 mL of polyethylenimine (linear, MW of approximately 423) (3.6 mmoles) dissolved in 50 mL pyridine was added dropwise over a period of 30 minutes. The reaction was allowed to proceed for 72 hours at RT and then concentrated to about 25 mL in a rotary evaporator. To the resulting suspension, 50 mL of distilled water was added, the suspension was stirred overnight, and the precipitated material removed by filtration. The clear filtrate was evaporated in a rotary evaporator to remove the pyridine. The syrupy residue was dissolved in 15 mL of distilled water, filtered and dialyzed for 3 days against water using a membrane with a molecular weight cut off of 3500. The dialyzed material was filtered to remove any insoluble matter, frozen in a dry ice/isopropanol bath and freeze-dried to recover 1.43 g of the polyethylenimine quillaja saponin derivative. The derivative was analyzed by HPLC using a Vydac C4 column.

### Example 6

### High Performance Liquid Chromatography (HPLC) Purification of Saponin Derivatives

Final preparations of small molecular weight derivatives, molecular weights up to 5000, (˜100 to 20 μg) were analyzed by reverse phase HPLC using a Vydac C4 column (5 μm particle size, 300 Å pore size, 0.46×25 cm), eluted with a water/acetonitrile linear gradient between 10 to 40% acetonitrile and using a flow rate of 1 mL/min. Under certain conditions, the eluent contained 0.1% diethylamine to limit the ionization of the cationic groups of the derivatives. Effluent was monitored at 214 nm.

### Example 7

### Chromatographic Analysis of High Molecular Weight Glycoside/saponin-Polymer Conjugates

Glycoside/saponin conjugates containing high molecular weight polymers, such as proteins, polylysine, and similar cationic polymers were analyzed by one of the following procedures:

i) Sodium dodecyl sulfate (SDS) polyacrylamide gel electrophoresis, using a gel containing 8-10% acrylamide, 0.1% SDS and 0.1 M Na phosphate pH ˜7.

ii) gel filtration using 6 M urea/0.5 M acetic acid or 50% dimethylsulfoxide as an eluent.

iii) ion-exchange chromatography on a carboxymethylated matrix using a NaCl salt gradient in 6 M urea at pH ˜4.50

### Example 8

### Testing of Immune Stimulatory Effect on DNA Vaccines Using a DNA Plasmid for OVA

The immune stimulatory effect of a compound over the immune response elicited by DNA vaccination can be assessed by the antibody response against a transiently expressed antigen encoded by a DNA or RNA sequence. An indication of the modulatory effects of a compound on the type of immune response can be obtained from the stimulation of the different antibody isotypes. In effect, production in mice of the IgG2a isotype has been associated with Th1 immunity, while a predominant IgG1 response is a good indicator of Th2 immunity.

The immune stimulatory effect of some compounds was determined by the increase of anti-OVA antibodies after immunization with a DNA plasmid for OVA in the presence and absence of such compounds. Female BALB/c mice of approximately 6 to 9 weeks of age were immunized intramuscularly on days 1 and 15 with 50 or 100 μg of the compounds being tested. Injections were given in two sites (50 μL/site) in a total volume of 100 μL. Mice injected with PBS only were used as negative controls. Sera was collected on days 29, 50 and 71 and assayed for anti-OVA antibodies by ELISA using Immunion II plates coated overnight at 4° C. with 100 μL per well of an OVA solution (50 μg/mL). Plates were washed twice with PBS and non-specific binding prevented by incubating all the wells for 1.5 hour at 37° C. with 100 μL of 2% casein hydrolysate in PBS. Plates were washed 4 times with 0.05% Tween 20 in distilled water. The initial sera dilution used was 1:30 and samples were diluted serially 1:2 thereafter. Sera dilutions were incubated for 1.5 hours at 37° C., plates washed and incubated with anti-IgG-HPR conjugates, washed and developed with a TMB substrate for 15 minutes at room temperature, and the reaction stopped by addition of 0.18 M sulfuric acid. Titers were determined at 450 nm using a cut-off value of 0.1 O.D.

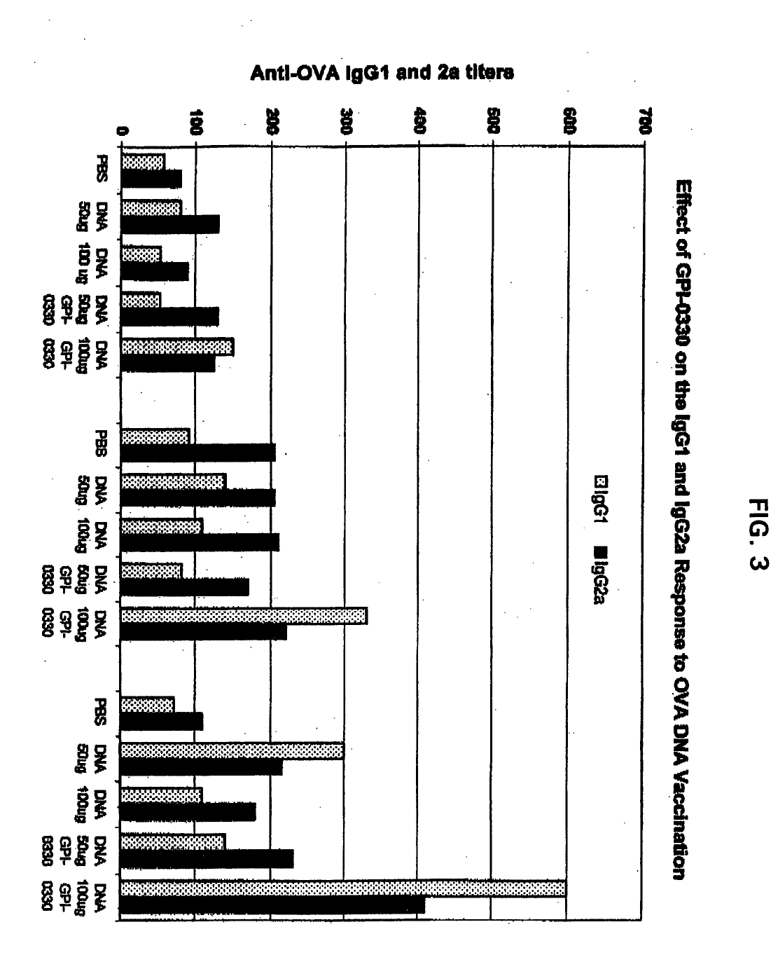

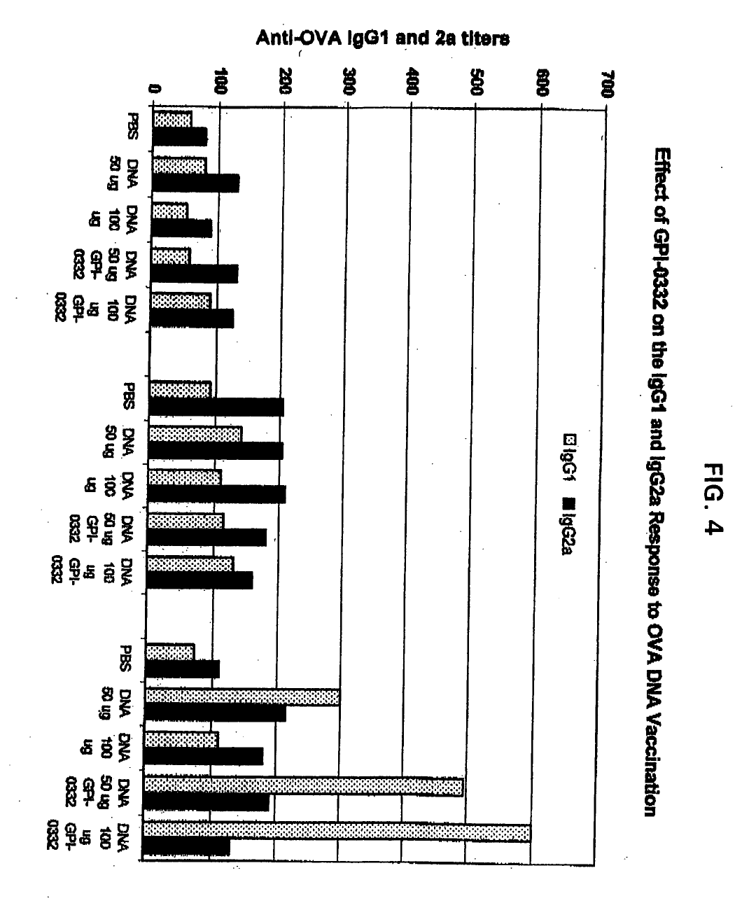

FIGS. 3 and 4 illustrate the results of use of this protocol to measure the immune stimulatory effect of GPI-0330 and GPI-0332 on the IgG1 and IgG2a production in BALB/c mice.

### Example 9

### Immunization of Balb/c Mice with OVA cDNA in the Presence and Absence of 3-dimethylamino-1-propylamino-DS-saponin (DMPS)

Female Balb/c mice were immunized intramuscularly at days 1 and 15 with 0.2 mL of phosphate buffered saline solution (PBS) containing 20 μg of chicken OVA cDNA alone or with 50 μg of 3-dimethylamino-1-propylamino-DS-saponin (DMPS). The complete OVA cDNA was sub-cloned into a mammalian expression vector containing the human β-actin promotor and the neomycin resistant gene, under control of the SV40 promotor, to yield pAC-Neo-OVA. Negative control animals received PBS only. Animals were bled 14 days after the last immunization. Total IgG and IgG2a were determined by ELISA using OVA coated plates and a serum dilution of 1:100. The serum dilution was incubated at 37° C. for 1 hour and the plates were washed. After incubation with anti IgG-HRP conjugate, the plates were washed and developed with a TMB substrate for 15 minutes at room temperature, stopped by the addition of 0.18 M sulfuric acid, and read at 450 nm.

Results

| | | Absorbance (450 nm) | 20 mg OVA DNA |
|---|---|---|---|
| | PBS (−) | 20 mg OVA DNA | +50 mg DMPS |
| 1- | 0.128 | 0.144 | 0.400 |
| 2- | 0.089 | 0.080 | 0.210 |
| 3- | 0.072 | 0.100 | 0.092 |
| 4- | 0.084 | 0.096 | 0.140 |
| 5- | 0.112 | 0.124 | 0.100 |
| Average: | 0.097 ± 0.018 | 0.109 ± 0.020 | 0.188 ± 0.093 |

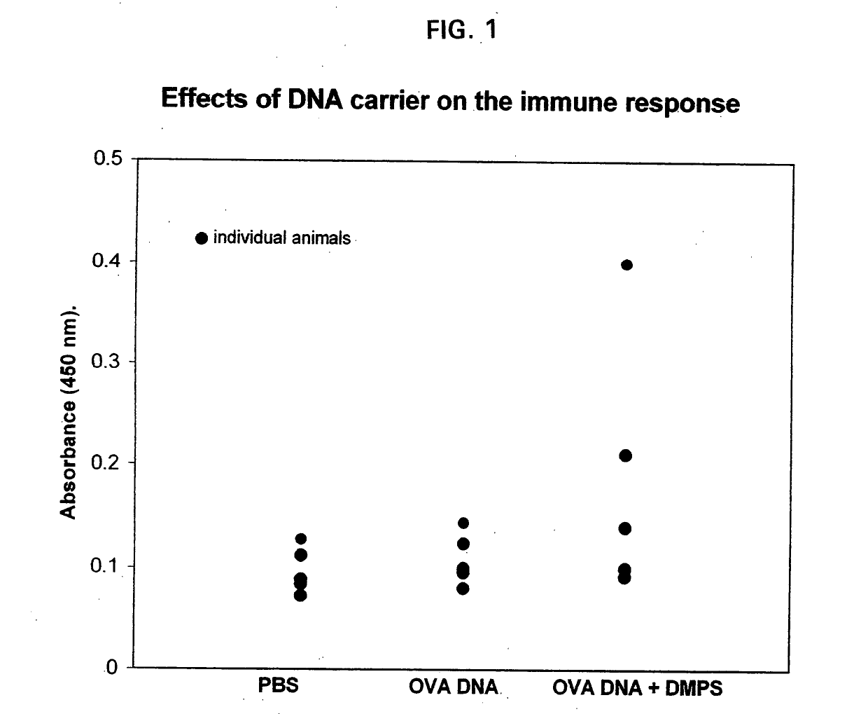

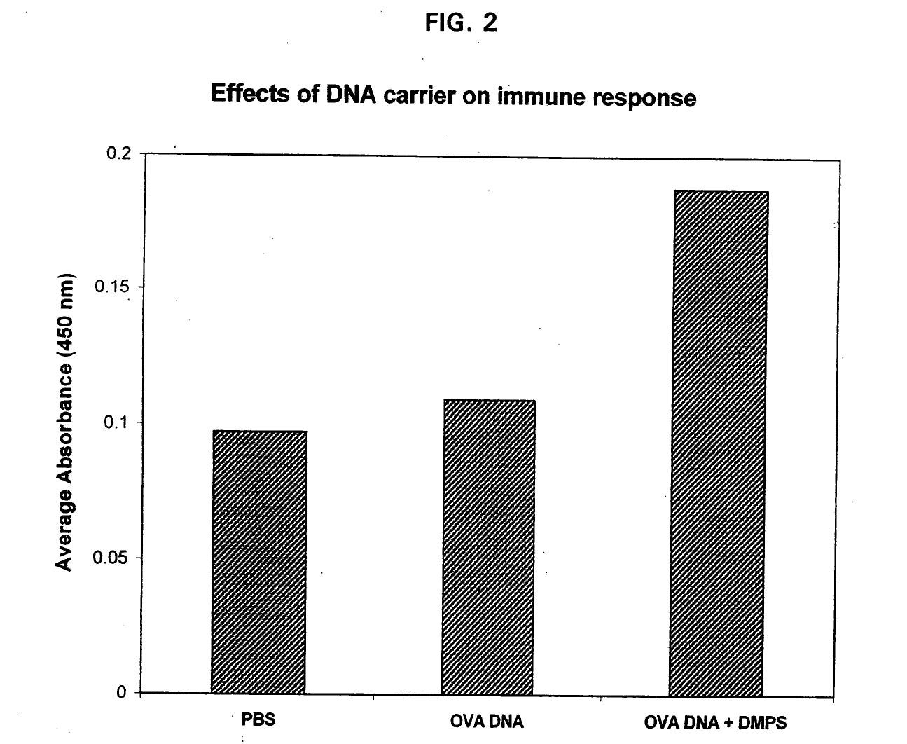

The results, illustrated in FIGS. 1 and 2, demonstrate that the saponin derivatives of the present invention, when co-administered with a nucleic acid encoding for an antigen, stimulate the immune response in mice by stimulating antibody production.

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

What is claimed is:

1. A saponin compound comprising:
(a) an aglycone core, wherein said aglycone core is covalently linked to one or more oligosaccharide chains; and
(b) a positively charged cationic chain, wherein said chain comprises
(i) three or more carbon atoms; and
(ii) one or more primary amine groups, one or more secondary amine groups, one or more tertiary amine groups, or one or more guanidine groups, or any combination thereof;
and wherein said positively charged cationic chain is covalently bound to said compound.

2. The compound of claim 1, further comprising:
(c) a lipophilic chain, wherein said lipophilic chain comprises from 4 to 36 carbon atoms and optionally contains one or more oxyethylene groups.

3. The compound of claim 1, wherein said aglycone core is a triterpenoid aglycone core.

4. The compound of 3, wherein said one or more oligosaccharide chains are covalently linked at C-3, at C-28, or at both C-3 and C-28, of said aglycone core.

5. The compound of claim 1, wherein said compound contains an aldehyde group or a ketone group that is capable of forming an imine or Schiff base with an amino group of a receptor protein on the surface of a cell.

6. The compound of claim 5, wherein said aldehyde or ketone group is attached to said aglycone core.

7. The compound of claim 5, wherein said aldehyde or ketone group is attached to one or more of said oligosaccharide chains.

8. The compound of claim 1, wherein said positively charged cationic chain is selected from the group consisting of a linear aliphatic chain, a branched aliphatic chain, an oligosaccharide, a polysaccharide, a protein, and a polypeptide that is cationic or has been subsequently modified by the introduction of amino groups or similar cationic basic groups that are capable of forming a complex with DNA or RNA.

9. The compound of claim 8, wherein said positively charged cationic chain is a protein.

10. The compound of claim 9, wherein said protein is a histone or protamine.

11. The compound of claim 1, wherein said cationic chain has a molecular weight ranging from 100 daltons to 100,000 daltons.

12. The compound of claim 1, wherein said cationic chain is attached either to a sugar residue of said oligosaccharide chain or to said aglycone core.

13. The compound of claim 2, wherein said lipophilic chain is attached to a sugar residue of said oligosaccharide chain or to said aglycone core.

14. A saponin derivative/polynucleotide complex formed by the association of a compound of claim 1 with a polynucleotide.

15. The complex of claim 14, wherein said polynucleotide is a DNA polynucleotide.

16. The complex of claim 15, wherein said DNA polynucleotide is a noncoding bacterial DNA polynucleotide.

17. The complex of claim 15, wherein said DNA polynucleotide encodes an immunogen.

18. A saponin derivative/polynucleotide secondary complex formed by association of the saponin derivative/polynucleotide complex of claim 14 with one or more saponins selected from the group consisting of a native saponin, a semi-synthetic saponin derivative, and a synthetic saponin containing a triterpenoid aglycone core covalently linked to one or more oligosaccharide chains.

19. A pharmaceutical composition comprising a compound of claim 1 and a pharmaceutically acceptable carrier or diluent.

20. A pharmaceutical composition comprising:
(c) a compound of claim 1;
(d) a polynucleotide encoding an immunogen, wherein said polynucleotide is operably linked to a promoter; and
(e) a pharmaceutically acceptable carrier or diluent.

21. A pharmaceutical composition comprising:
(a) a compound of claim 1;
(b) a noncoding bacterial DNA polynucleotide; and
(c) a pharmaceutically acceptable carrier or diluent.

22. A pharmaceutical composition comprising a saponin derivative/polynucleotide complex of claims 14 or 15 and a pharmaceutically acceptable carrier or diluent.

23. A method of delivering a polynucleotide to cells of an animal in need thereof, comprising administration in vivo to the animal of a polynucleotide construct comprising a polynucleotide sequence encoding an immunogen, and a compound of claim 1.

24. The method of claim 23, wherein said polynucleotide construct forms a complex with said compound of claim 1.

25. The method of claim 23, wherein the animal is human.

26. The method of claim 23, wherein said polynucleotide sequence is a DNA sequence that is operably linked to a promoter.

27. The method of claim 23, wherein said polynucleotide sequence is mRNA.

28. The method of claim 23, wherein said construct is a plasmid DNA.

29. The method of claim 23, wherein said administration is intravenous, intramuscular, subcutaneous, transdermal, intranasal, or transmucosal.

30. A method of delivering a polynucleotide to cells of an animal in need thereof, comprising:
(a) forming a saponin derivative/nucleic acid complex, wherein said complex is formed by association of a compound of claim 1 and a polynucleotide sequence encoding an immunogen; and
(b) administering said complex in vitro to the cells of the animal in an amount sufficient that uptake of said polynucleotide sequence into the cells of the animal occurs.

31. A method of stimulating an immune response in an animal in need thereof, comprising
administering in vivo to the animal a noncoding bacterial DNA polynucleotide and a compound of claim 1.

32. The method of claim 31, wherein said noncoding bacterial DNA polynucleotide forms a complex with said compound of claim 1.

33. The method of claim 31, said method further comprising administering in vivo to the animal a polypeptide antigen.

34. The method of claim 31, said method further comprising administering in vivo to the animal a polynucleotide sequence encoding an immunogen.

35. A method of stimulating an immune response in an animal in need thereof, comprising
administering in vivo to the animal a polynucleotide sequence encoding an immunogen, wherein said polynucleotide sequence is operably linked to a promoter, and a compound of claim 1;
wherein said polynucleotide sequence is administered to the animal in an amount sufficient that uptake of said polynucleotide sequence into cells of the animal occurs, and sufficient expression results, to stimulate the immune response in the animal.

36. The method of claim 35, wherein said polynucleotide sequence forms a complex with said compound of claim 1.

37. A method of stimulating an immune response in an animal in need thereof, comprising:
(a) introducing into the cells of the animal a polynucleotide sequence encoding an immunogen, wherein said polynucleotide sequence is operably linked to a promoter, and a compound of claim 1; and
(b) introducing the cells into the animal, wherein sufficient expression of the immunogen occurs in the cells and an immune response is stimulated in the animal.

38. The method of claim 37, wherein said polynucleotide sequence forms a complex with said compound of claim 1.

39. A method of generating a detectable immune response in an animal in need thereof, comprising:
administering in vivo to the cells of an animal a polynucleotide sequence encoding an immunogen, wherein said polynucleotide sequence is operably linked to a promoter, and a compound of claim 1;
wherein said polynucleotide sequence is administered in an amount sufficient that uptake of said polynucleotide sequence into the cells of the animal occurs, and sufficient expression results, to generate the detectable immune response.

40. The method of claim 39, wherein said polynucleotide sequence forms a complex with said compound of claim 1.

41. A method of generating a detectable immune response in an animal in need thereof, comprising:
(a) introducing into the cells of the animal a polynucleotide sequence encoding an immunogen, wherein said polynucleotide sequence is operably linked to a promoter, and a compound of claim 1; and
(b) introducing the cells into the animal, wherein sufficient expression of the immunogen occurs in the cells and a detectable immune response if generated.

42. The method of claim 41, wherein said polynucleotide sequence forms a complex with said compound of claim 1.
